(19) United States
(12) Patent Application Publication
(10) Pub. No.: US 2022/0142955 A1
(43) Pub. Date: May 12, 2022

(54) METHODS OF TREATING ORGANIC ACIDEMIAS

(71) Applicant: HemoShear Therapeutics, Inc., Charlottesville, VA (US)

(72) Inventors: Brian WAMHOFF, Charlottesville, VA (US); John REARDON, Charlottesville, VA (US); Robert FIGLER, Charlottesville, VA (US)

(21) Appl. No.: 17/438,560

(22) PCT Filed: Mar. 13, 2020
(86) PCT No.: PCT/US2020/022760

§ 371 Date: Sep. 13, 2021

Related U.S. Application Data
(60) Provisional application No. 62/818,372, filed on Mar. 14, 2019.

(51) Int. Cl. A61K 31/185; A61P 3/00

(52) U.S. Cl. CPC A61K 31/185; A61P 3/00

(57) ABSTRACT

The present disclosure relates to methods of treating organic acidemias. In some embodiments, the methods comprise reducing propionyl-CoA, isovaleryl-CoA and methylmalonyl-CoA production, and various related metabolites, in a subject in need thereof.

### FIELD OF THE INVENTION

The present disclosure relates to novel therapeutic strategies for treating metabolic disorders.

### BACKGROUND

Metabolic disorders occur when there is a mutation in an enzyme that causes a significant loss of function which interrupts the normal flux of metabolites in a metabolic pathway. This results in accumulation of normal intermediary metabolites in abnormally large amounts and in some cases, the production of abnormal metabolites that are not normally formed when there is not a mutation that causes a significant loss of function in an enzyme.

For example, propionic acidemia (PA) and methylmalonic acidemia (MMA) are inborn errors of metabolism that result in the buildup of metabolites. The incidence rates for PA are 1 in 242,741 individuals in the US, 1 in 50,000 to 100,000 people worldwide, and the incidence can be as high as 1 in 1,000 to 2,000 in specific populations that are genetically at higher risk (e.g. Inuit population of Greenland, some Amish communities, Saudi Arabians, and communities with consanguineous marriage) whereas MMA affects 1 in 69,354 births.

PA is caused by a dysfunction of the propionyl-CoA carboxylase (EC 6.4.1.3) enzyme which blocks the conversion of propionyl-CoA to methylmalonyl-CoA resulting in the accumulation of propionyl-CoA in cells and metabolites such as 3-hydroxypropionic acid, 2-methylcitric acid, and propionylcarnitine in the urine and in the blood. Inhibition of the urea cycle (assumed to be by 3-hydroxypropionic acid or propionyl-CoA) results in clinically significant elevations in blood ammonia, contributing to both morbidity and mortality.

MMA is caused by dysfunction of the vitamin B12-dependent methylmalonyl-CoA mutase (EC 5.4.99.2) enzyme, which blocks the conversion of methylmalonyl-CoA to succinyl-CoA resulting in the accumulation of metabolites such as propionyl-CoA, methylmalonyl-CoA, methylmalonic acid, 3-hydroxypropionic acid, 2-methylcitric acid, and propionylcarnitine in the blood and tissues. A complete or partial enzyme deficiency results in the mut⁰ or mut⁻ disease subtype, respectively. In some instances, MMA can be caused by a dysfunction of the methylmalonyl-CoA epimerase (EC 5.1.99.1) enzyme, also called methylmalonyl racemase. In addition, MMA can also be caused by defective synthesis of adenosylcobalamin (an active form of vitamin B12) by MMAA, MMAB and MMADHC. Similar to PA, the accumulation of certain toxic metabolites in MMA patients results in reduced urea cycle function (assumed to be by 3-hydroxypropionic acid or propionyl-CoA), which can cause clinically significant elevations in blood ammonia, contributing to both morbidity and mortality.

Patients suffering from PA or MMA have elevated levels of certain metabolites resulting from defective enzymes (propionyl-CoA carboxylase or methylmalonyl-CoA mutase, respectively). Patients with PA and MMA often present acutely with metabolic acidosis, dehydration, lethargy, seizures, vomiting, and hyperammonemia causing severe central nervous system dysfunction. Long term complications include seizures, cardiomyopathies, metabolic stroke like episodes, cardiac arrhythmias, chronic kidney failure, impaired consciousness, ketosis, pancreatitis and optic atrophy, which severely impact the quality of life and cause progressive deterioration, sometimes ending in sudden death.

There are no current definitive therapies for PA or MMA. Mostly, treatment options focus on severe dietary and lifestyle modifications and symptomatic management of the complications and sequelae arising due to acute and long-term exposure to toxic metabolites associated with the disease state. The dietary regimen involves restricting the precursors of propionyl-CoA, such as branched-chain amino acids (valine and isoleucine), threonine, methionine, odd-chain fatty acids and cholesterol, while trying to maintain normal growth. Dietary supplementation with levocarnitine, biotin (PA) and/or cobalamin (MMA) is also common. In addition, propiogenic gut bacteria is controlled with antibiotic regimens, and complications are treated symptomatically as they occur. Despite the symptomatic relief, many of these patients still progress to the long-term sequelae of the disease.

Liver and/or kidney transplantation may be required. For example, some patients with PA receive orthotopic liver transplantation (OLT) to ameliorate symptoms primarily due to hyperammonemia.

Therefore, developing an effective therapeutic method to treat PA and MMA is critical for improving clinical manifestations of the disease as well as improving the quality of life and life span of these patients. Thus, there exists a need to treat metabolic disorders (e.g., PA and MMA) by reducing the levels of toxic metabolites associated with the disease state. The present disclosure solves this need.

### SUMMARY OF DISCLOSURE

In some embodiments, the present disclosure provides methods of treating an organic acidemia (e.g., propionic acidemia (PA), isovaleric acidemia (IVA), or methylmalonic acidemia (MMA), or any other disease disclosed herein) in a subject in need thereof, comprising administering to a patient one or more compounds of Formula I thereby reducing levels of propionyl-CoA, isovaleryl-CoA, methylmalonyl-CoA, or a combination thereof in the patient. In some embodiments, the compound of Formula I has a structure according to Formula IA, or Formula II, or Formula IIA. In some embodiments, the compound of Formula I, Formula IA, or Formula II is 2,2-dimethylbutyric acid (also referred to as 2,2-dimethylbutanoic acid), or a metabolite, ester, or pharmaceutically acceptable salt thereof.

In some embodiments, the present disclosure provides methods of reducing propionyl-CoA or methylmalonyl-CoA, isovaleryl-CoA, or a combination thereof, in a subject in need thereof comprising administering one or more compounds of Formula I, or a coenzyme-A ester or carnitine ester thereof, or a pharmaceutically acceptable ester, solvate, or salt thereof. In some embodiments, the compound of Formula I has a structure according to Formula IA, II, or HA. In some embodiments, the compound of Formula I, IA, or II is 2,2-dimethylbutyric acid, a coenzyme A ester or carnitine ester thereof, or a pharmaceutically acceptable metabolite, ester, solvate, or salt thereof.

In some embodiments, the compounds of the disclosure (e.g., Formula I, IA, II, and/or HA) are formulated in pharmaceutical compositions. In some embodiments, the pharmaceutical composition comprises a pharmaceutically acceptable carrier or a pharmaceutically acceptable excipient. In some embodiments, the compounds of the disclosure are administered orally.

In some embodiments, methods comprise reducing production of at least one metabolite that otherwise accumulates to toxic levels in patients with a metabolic disorder (including organic acidemias, e.g., IVA, PA and/or MMA). In some embodiments, at least one metabolite is reduced by least about 1% to about 1000%, e.g., about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, and 100%, inclusive of all values and subranges therebetween. In some embodiments, the metabolite is a metabolite of one or more of the branched-chain amino acids, methionine, threonine, odd-chain fatty acids and cholesterol. In some embodiments, the metabolite is propionic acid, 3-hydroxypropionic acid, 2-methylcitrate, methylmalonic acid, propionylglycine, or propionylcarnitine, or combinations thereof. In some embodiments, the at least one metabolite comprises 2-ketoisocaproate, isovaleryl-CoA, 3-methylcrotonyl-CoA, 3-methylglutaconyl-CoA, 3-OH-3-methylglutaryl-CoA, 2-keto-3-methylvalerate, 2-methylbutyryl-CoA, tiglyl-CoA, 2-methyl-3-OH-butyryl-CoA, 2-methyl-acetoacetyl-CoA, 2-ketoisovalerate, isobutyryl-CoA, methylacrylyl-CoA, 3-OH-isobutyryl-CoA, 3-OH-isobutyrate, methylmalonic semialdehyde, propionyl-CoA, or methylmalonyl-CoA, or combinations thereof. In some embodiments, the amount of propionyl-CoA produced is reduced by at least about 1% to about 100%. In some embodiments, the amount of methylmalonyl-CoA produced is reduced by at least about 1% to about 100%.

### BRIEF DESCRIPTIONS OF THE FIGURES

FIG. 1 shows the effects of compounds on the concentrations of ¹³C-propionyl-CoA and the ¹²C-Compound-CoA esters in the presence of ¹³C-labeled isoleucine in primary hepatocytes of propionic acidemia patients. All primary hepatocytes were treated with compounds ranging from 0 μM to 1,000 μM. FIG. 1A shows the concentration of ¹³C-propionyl-CoA in primary hepatocytes treated with compound 1. The concentration of ¹³C-propionyl-CoA had an EC₅₀ of 12.43 μM and the concentration of ¹²C-Compound 1-CoA ester had an EC₅₀ of 12.47 μM. FIG. 1B shows the concentration of ¹³C-propionyl-CoA in primary hepatocytes treated with compound 2. The concentration of ¹³C-propionyl-CoA had an EC₅₀ of 1.23 μM and the concentration of ¹²C-Compound 2-CoA ester had an EC₅₀ of 1.24 μM. FIG. 1C shows the concentration of ¹³C-propionyl-CoA in primary hepatocytes treated with compound 3. The concentration of ¹³C-propionyl-CoA had an EC₅₀ of 13.04 μM and the concentration of ¹²C-Compound 3-CoA ester had an EC₅₀ of 27.41 μM. FIG. 1D shows the concentration of ¹³C-propionyl-CoA in primary hepatocytes treated with compound 4. The concentration of ¹³C-propionyl-CoA had an EC₅₀ of 32.4 μM and the concentration of ¹²C-Compound 4-CoA ester had an EC₅₀ of 10.29 μM. FIG. 1E shows the concentration of ¹³C-propionyl-CoA in primary hepatocytes treated with compound 5. The concentration of ¹³C-propionyl-CoA had an EC₅₀ of 0.43 μM and the concentration of ¹²C-Compound 5-CoA ester had an EC₅₀ of 0.95 μM. FIG. 1F shows the concentration of ¹³C-propionyl-CoA in primary hepatocytes treated with compound 6. The concentration of ¹³C-propionyl-CoA had an EC₅₀ of 0.91 μM and the concentration of ¹²C-Compound 6-CoA ester had an EC₅₀ of 0.48 μM. FIG. 1G shows the concentration of ¹³C-propionyl-CoA in primary hepatocytes treated with compound 7. The concentration of ¹³C-propionyl-CoA had an EC₅₀ of 28.79 μM and the concentration of ¹²C-Compound 7-CoA ester had an EC₅₀ of 10.15 μM.

FIG. 2 shows the effects of compound 1 on the concentrations of propionyl-CoA from various sources in primary hepatocytes of propionic acidemia patients. All primary hepatocytes were treated with compound 1 ranging from 0 μM to 1,000 μM. FIG. 2A shows the concentration of propionyl-CoA in primary hepatocytes in the presence of 1 mM ¹C-KIVA (ketoisovaleric acid). The concentration of propionyl-CoA had an EC₅₀ of 14.17 μM. FIG. 2B shows the concentration of propionyl-CoA in primary hepatocytes in the presence of 3 mM ¹³C-ILE (isoleucine). The concentration of propionyl-CoA had an EC₅₀ of 15.01 μM. FIG. 2C shows the concentration of propionyl-CoA in primary hepatocytes in the presence of 5 mM ¹³C-THR (threonine). The concentration of propionyl-CoA had an EC₅₀ of 9.2 μM. FIG. 2D shows the concentration of propionyl-CoA in primary hepatocytes in the presence of 5 mM ¹³C-MET (methionine). The concentration of propionyl-CoA had an EC₅₀ of 7.14 μM. FIG. 2E shows the concentration of propionyl-CoA in primary hepatocytes in the presence of 5 mM ¹³C-propionate. The concentration of propionyl-CoA had an EC₅₀ of 21.18 μM. FIG. 2F shows the concentration of propionyl-CoA in primary hepatocytes in the presence of 100 μM ¹³C-heptanoate. The concentration of propionyl-CoA had an EC₅₀ of 48.2 μM.

FIG. 3 shows the effects of compound 5 on the concentrations of propionyl-CoA from various sources in primary hepatocytes of propionic acidemia patients. All primary hepatocytes were treated with compound 5 ranging from 0 μM to 1,000 μM. FIG. 3A shows the concentration of propionyl-CoA in primary hepatocytes in the presence of 1 mM ¹³C-KIVA. The concentration of propionyl-CoA had an EC₅₀ of 0.89 μM. FIG. 3B shows the concentration of propionyl-CoA in primary hepatocytes in the presence of 3 mM ¹³C-ILE. The concentration of propionyl-CoA had an EC₅₀ of 0.42 μM. FIG. 3C shows the concentration of propionyl-CoA in primary hepatocytes in the presence of 5 mM ¹³C-THR. The concentration of propionyl-CoA had an EC₅₀ of 1.24 μM. FIG. 3D shows the concentration of propionyl-CoA in primary hepatocytes in the presence of 5 mM ¹³C-propionate. The concentration of propionyl-CoA had an EC₅₀ of 15.27 μM.

FIG. 4 shows the effects of compound 1 on the concentrations of propionyl-CoA and methylmalonyl-CoA from various sources in primary hepatocytes of methylmalonic acidemia patients. All primary hepatocytes were treated with compound 1 ranging from 0 μM to 1,000 μM. FIG. 4A shows the concentrations of propionyl-CoA and methylmalonyl-CoA in primary hepatocytes in the presence of 1 mM ¹C-KIVA. The concentration of propionyl-CoA had an EC₅₀ of 30.9 μM. The concentration of and methylmalonyl-CoA had an EC₅₀ of 31.26 μM. FIG. 4B shows the concentrations of propionyl-CoA and methylmalonyl-CoA in primary hepatocytes in the presence of 3 mM ¹³C-ILE. The concentration of propionyl-CoA had an EC₅₀ of 30.79 μM. The concentration of and methylmalonyl-CoA had an EC₅₀ of 25.53 μM. FIG. 4C shows the concentrations of propionyl-CoA and methylmalonyl-CoA in primary hepatocytes in the presence of 5 mM ¹³C-THR. The concentration of propionyl-CoA had an EC₅₀ of 13.89 μM. The concentration of and methylmalonyl-CoA had an EC₅₀ of 25.58 μM. FIG. 4D shows the concentrations of propionyl-CoA and methylmalonyl-CoA in primary hepatocytes in the presence of 5 mM ¹³C-MET. The concentration of propionyl-CoA had an EC₅₀ of 50.71 μM. The concentration of and methylmalonyl-CoA had an EC₅₀ of 47.26 μM. FIG. 4E shows the concentrations of propionyl-CoA and methylmalonyl-CoA in primary hepatocytes in the presence of 100 μM ¹³C-propionate. The concentration of propionyl-CoA had an EC₅₀ of 68.25 μM. The concentration of and methylmalonyl-CoA had an EC₅₀ of 89.36 μM.

FIG. 5 shows the effects of compound 5 on the concentrations of propionyl-CoA and methylmalonyl-CoA from various sources in primary hepatocytes of methylmalonic acidemia patients. All primary hepatocytes were treated with compound 5 ranging from 0 μM to 1,000 μM. FIG. 5A shows the concentrations of propionyl-CoA and methylmalonyl-CoA in primary hepatocytes in the presence of 1 mM ¹³C-KIVA. The concentration of propionyl-CoA had an EC₅₀ of 0.93 μM. The concentration of and methylmalonyl-CoA had an EC₅₀ of 1.17 μM. FIG. 5B shows the concentrations of propionyl-CoA and methylmalonyl-CoA in primary hepatocytes in the presence of 3 mM ¹³C-ILE. The concentration of propionyl-CoA had an EC₅₀ of 2.04 μM. The concentration of and methylmalonyl-CoA had an EC₅₀ of 1.38 μM. FIG. 5C shows the concentration of propionyl-CoA in primary hepatocytes in the presence of 100 μM ¹³C-heptanoate. The concentration of propionyl-CoA had an EC₅₀ of 3.84 μM. The concentration of methylmalonyl-CoA had an EC₅₀ of 0.02 μM.

FIG. 6 shows the effects of compound 1 on the concentrations of propionyl-carnitine from various sources in primary hepatocytes of propionic acidemia patients. All primary hepatocytes were treated with compound 1 ranging from 0 μM to 1,000 μM. FIG. 6A shows the concentration of propionyl-carnitine in primary hepatocytes in the presence of 1 mM ¹³C-KIVA. The concentration of propionyl-carnitine had an EC₅₀ of 44.33 μM. FIG. 6B shows the concentration of propionyl-carnitine in primary hepatocytes in the presence of 3 mM ¹³C-ILE. The concentration of propionyl-carnitine had an EC₅₀ of 54.26 μM.

FIG. 7 shows representative activity data from PA donor 1 and MMA donor 1 in the HemoShear Technology upon treatment of primary hepatocytes with Compound 5. FIG. 7A shows the dose-dependent reduction of propionyl-CoA (“P-CoA”) in PA and MMA primary hepatocytes. FIG. 7B shows the dose-dependent reduction in methylmalonyl (“M-CoA”) (labeled with ¹³C in MMA primary hepatocytes. FIG. 7C shows the dose-dependent reduction of propionyl-carnitine (C3) concentration in PA and MMA primary hepatocytes. FIG. 7D shows the dose-dependent reduction of the propionyl-carnitine/acetyl-carnitine (C3/C2) ratio in PA and MMA primary hepatocytes. FIG. 7E shows the dose-dependent reduction of MCA concentration in PA and MMA primary hepatocytes.

FIG. 8 shows dose-response curves for the treatment of PA and MMA primary hepatocytes in static cell culture using from 0.1 μM to 100 μM concentrations of Compound 5. FIG. 8A shows the intracellular concentration of ¹³C-P-CoA in PA and MMA primary hepatocytes treated with Compound 5 under low and high propiogenic conditions. FIG. 8B shows the intracellular concentration of ¹³C-M-CoA in PA and MMA primary hepatocytes treated with Compound 5 under low and high propiogenic conditions. FIG. 8C shows the intracellular concentration of ¹³C-methylmalonic acid in MMA primary hepatocytes treated with Compound 5 under low and high propiogenic conditions.

FIG. 9 shows the pharmacology of Compound 5 in static cell culture in PA primary hepatocytes and MMA primary hepatocytes, under low and high propiogenic conditions. FIG. 9A shows effects of Compound 5 on ¹³C-P-CoA levels measured in PA and MMA pHeps in static cell culture experiments. FIG. 9B shows the effects of Compound 5 on acetyl-CoA levels measured in PA and MMA pHeps in static cell culture experiments. FIG. 9C shows the effects of Compound 5 on CoASH levels measured in PA and MMA pHeps in static cell culture experiments. FIG. 9D shows the dose-dependent increase in Compound 5-CoA formation when PA and MMA pHeps were exposed to Compound 5 for 1.5 h.

FIG. 10 shows the pharmacology of Compound 5 in the HemoShear Technology in PA primary hepatocytes, MMA primary hepatocytes, and normal primary hepatocytes. FIG. 10A shows effects of Compound 5 on ¹³C-P-CoA levels measured in PA and MMA pHeps exposed to Compound 5 for 6 days. FIG. 10B shows the effects of Compound 5 on acetyl-CoA levels measured in PA and MMA pHeps exposed to Compound 5 for 6 days. FIG. 10C shows the effect on CoASH levels measured in PA, MMA, and normal pHeps exposed to Compound 5 for 6 days. FIG. 10D shows the dose-dependent increase in Compound 5-CoA formation when PA and MMA pHeps were exposed to Compound 5 for 6 days.

FIG. 11 provides a schematic of HemoShear Technology. In FIG. 11A, Primary hepatocytes were maintained in a system modeled based on the sinusoidal configuration under conditions that maintain the physiological hemodynamics and transport, and have shown to retain and restore liver like phenotype, morphology, function and responses. FIG. 11B shows a cross section of the HemoShear Technology,

### DETAILED DESCRIPTION

### Definitions

Unless otherwise defined, all terms used in this application should be given their standard and typical meanings in the art and are used as those terms would be used by a person of ordinary skill in the art at the time of the invention.

In this application, including the appended claims, the singular forms “a,” “an,” and “the” are often used for convenience. However, it should be understood that these singular forms include the plural unless otherwise specified.

When a numerical range is disclosed herein, it is to be understood that all values and subranges therein are included as if each was expressly disclosed. For example, a range of from about 1 to about 100 is understood to include all values between 1 and 100, e.g., 2, 3, 4, 5, 6, 7, 8, 9, 10, 15, 20, 25, 30, 35, 40, 45, 50, 55, 60, 65, 70, 75, 80, 85, 90, 95, and 99 inclusive of all values and subranges therebetween. As an additional example, a range of from about 1 to about 100 is understood to include all subranges within the range, e.g., 1-42, 37-100, 25-65, 75-98, etc.

Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₂ alkenyl include ethynyl, propynyl, butynyl, pentynyl and the like. Unless stated otherwise specifically in the specification, an alkyl group can be optionally substituted.

The term “alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Carbocyclyl,” “carbocyclic ring” or “carbocycle” refers to a ring structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Carbocyclylalkyl” refers to a radical of the formula —R_{b}—R_{d} where R_{b} is an alkylene, alkenylene, or alkynylene group as defined above and Rd is a carbocyclyl radical as defined above. Unless stated otherwise specifically in the specification, a carbocyclylalkyl group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Arylalkyl” refers to a radical of the formula —R_{b}—_{Rd} where R_{b} is an alkylene, alkenylene, or alkynylene group as defined above and Rd is an aryl radical as defined above. Unless stated otherwise specifically in the specification, an arylalkyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

Heterocyclyl,” “heterocyclic ring” or “heterocycle” refers to a stable 3- to 20-membered aromatic or non-aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylalkyl” refers to a radical of the formula —R_{b}—Rₑ where R_{b} is an alkylene, alkenylene, or alkynylene group as defined above and Rₑ is a heterocyclyl radical as defined above. Unless stated otherwise specifically in the specification, a heterocyclylalkyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a deuterium; a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

The term “leaving group” as used herein refers to an atom or group of atoms that departs with a pair of electrons in heterolytic bond cleavage. In some embodiments, the leaving group is an anion. In other embodiments, the leaving group is a neutral atom or group of atoms. Examples of anionic leaving groups include, but are not limited to halides (Cl⁻, Br⁻, I⁻), sulfonates (e.g., tosylate, mesylate, trifluomethylsulfonate), and carboxylates. Examples of neutral leaving groups include, but are not limited to water, ammonia, and tertiary amines (e.g., triethylamine). In some embodiments, the leaving group departs from a pharmaceutically acceptable core as part of a nucleophilic substitution pathway.

The term “pathway” or “metabolic pathway” refers to a series of biochemical or chemical reactions, catalyzed by enzymes that occur within a cell.

The term “metabolite” or variations thereof as used herein refers to molecules which are formed during metabolic processes. The term “metabolite” includes precursors, such as metabolic precursors, of biologically produced molecules and molecules which participate in a bio-chemical reaction to produce another compound. The term “metabolite” also includes the active moiety formed after administration and catabolism of the compound disclosed herein, e.g., 2-propylpentanoic acid or 2,2-dimethylbutanoic acid. For example, carnitine esters or coenzyme-A esters of 2,2-dimethylbutanoic acid may be formed at various stages of metabolism, and such esters may contribute to the therapeutic effect of the disclosed methods. As such, these metabolites are within the scope of the disclosure.

The term “metabolite that accumulates in organic acidemia patients” refers to metabolites that are present in aberrant levels in patients with an organic acidemia. To be clear, the term does not encompass a metabolite that is normally present at non-toxic levels in both healthy and organic acidemia patients. The term “metabolite that accumulates in propionic acidemia patients” as used herein refers to a metabolite of one or more of branched chain amino acid, methionine, threonine, odd-chain fatty acids, and cholesterol, wherein abnormal levels of said metabolite (compared to a healthy patient which does not have propionic acidemia) are characteristic of propionic acidemia. Similarly, the term “metabolite that accumulates in methylmalonic acidemia patients” as used herein refers to a metabolite of one or more of a branched chain amino acid, methionine, threonine, odd-chain fatty acids and cholesterol wherein abnormal levels of said metabolite (compared to a healthy patient which does not have methylmalonic acidemia) are characteristic of methylmalonic acidemia.

The term “enzyme” as used herein refers to any substance that catalyzes or promotes one or more chemical or biochemical reactions, which usually includes enzymes totally or partially composed of a polypeptide, but can include enzymes composed of a different molecule including polynucleotides.

The term “compound” as used herein means a molecule which is capable of reducing a particular metabolite associated with metabolic disorders. As used herein, a pharmaceutically acceptable compound includes its metabolites, salts, solvates, and prodrug thereof. For example, any reference to 2,2-dimethylbutyric acid expressly includes prodrugs, metabolites, salts, and solvates of 2,2-dimethylbutyric acid.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

The term “pharmaceutically acceptable esters” include those obtained by replacing a hydrogen on an acidic group with an alkyl group, for example by reacting the acid group with an alcohol or a haloalkyl group. Examples of esters include, but are not limited to, replacing the hydrogen on an —C(O)OH group with an alkyl to form an —C(O)Oalkyl.

The term “pharmaceutically acceptable solvate” refers to a complex of solute (e.g., active compound, salt of active compound) and solvent. If the solvent is water, the solvate may be referred to as a hydrate, for example, a mono-hydrate, a di-hydrate, a tri-hydrate, etc.

The term “pharmaceutically acceptable” as described herein is a material that is not biologically or otherwise undesirable, i.e., the material may be incorporated into a pharmaceutical formulation administered to a patient without causing any undesirable biological effects or interacting in a deleterious manner with any of the other components of the composition in which it is contained. When the term “pharmaceutically acceptable” is used to refer to a pharmaceutical excipient, such as a carrier, it is implied that the carrier or excipient has met the required standards of toxicological and manufacturing testing and/or that it is included on the Inactive Ingredient Guide prepared by the U.S. Food and Drug administration.

The term “effective amount” refers to an amount that effective for producing a therapeutic effect upon administration to a subject. The therapeutic effect can include treating a particular disease, such as, but not limited to, achieving a reduction in metabolite levels associated with an organic acidemia.

The term “administering” as used herein includes to any route of administration, for example, oral, parenteral, intramuscular, transdermal, intravenous, inter-arterial, nasal, vaginal, sublingual, subungual, etc. Administering can also include prescribing a drug to be delivered to a subject, for example, according to a particular dosing regimen, or filling a prescription for a drug that was prescribed to be delivered to a subject, for example, according to a particular dosing regimen.

The terms “treating” and “treatment” include the following actions: (i) preventing a particular disease or disorder from occurring in a subject who may be predisposed to the disease or disorder but has not yet been diagnosed as having it; (ii) curing, treating, or inhibiting the disease, i.e., arresting its development; or (iii) ameliorating the disease by reducing or eliminating symptoms, conditions, and/or by causing regression of the disease.

The terms “patient”, “subject” and “individual” are used interchangeably to refer to a human subject for whom or which therapy is desired, and generally refers to the recipient of the therapy to be practiced according to the invention.

### Metabolic Disorders

The present disclosure provides for methods of treating particular metabolic disorders that are characterized by the abnormal build-up of toxic metabolites of branched-chain amino acids. For example, inborn autosomal recessive metabolic disorders, such as PA and MMA are caused by enzyme activity deficiencies which result in the accumulation of metabolites of branched chain amino acids (e.g., valine and isoleucine), methionine, threonine, odd-chain fatty acids, or cholesterol, or combinations thereof. These diseases are classified as an organic acid disorder which is a condition that leads to an abnormal buildup of particular acids known as organic acids.

PA, an autosomal recessive metabolic disorder, is also known as propionic aciduria, propionyl-CoA carboxylase deficiency, or ketotic glycinemia. The disease is classified as an organic acid disorder which is a condition that leads to an abnormal buildup of particular acids known as organic acids. PA is caused by dysfunction of propionyl-CoA carboxylase (PCC), the heteropolymeric mitochondrial enzyme that catalyzes the conversion of propionyl-CoA to methylmalonyl-CoA. PCC is a heterododecamer (α6β6), comprising six α-subunits and six β-subunits (PCCA and PCCB, respectively). PCC is essential in the normal catabolism of branched-chain amino acids, threonine, methionine, odd-numbered chain length fatty acids, and cholesterol in the body.

PCC enzymatic activity deficiency results in accumulation of propionyl-CoA, propionyl-carnitine, propionyl-glycine, 3-hydroxy propionic acid, 2-methylcitric acid, glycine, ammonia (NH₃ and NH₄⁺) and lactate, among other metabolites in plasma and urine. PCC comprises alpha and beta subunits encoded by PCCA and PCCB, respectively. Different types of mutations can also lead to distinct disease phenotypes. For example, null alleles of PCCA (p.Arg313Ter, p.Ser562Ter) and PCCB (p.Gly94Ter) and several small deletions/insertions and splicing variants are associated with a more severe form of PA. Missense variants, in which partial enzymatic activity is retained (PCCA: p.Ala138Thr, p.Ile164Thr, p.Arg288Gly; PCCB: p.Asn536Asp), are associated with a milder phenotype. Exceptions may include the three PCCB missense variants p.Gly112Asp, p.Arg512Cys, and p.Leu519Pro, which affect heterododecamer formation and are associated with undetectable PCC enzyme activity and the severe phenotype. Other PCCB pathogenic variants such as p.Glu168Lys result in a wide variety of clinical manifestations among affected individuals. Additionally, in some examples, the PCCB pathogenic variant p.Tyr435Cys has been identified in asymptomatic children through newborn screening in Japan. Biallelic mutation of either PCCA or PCCB results in PA. 153 and 138 different types of mutations of PCCA and PCCB are discovered, respectively. For example, one mutation of a subunits of propionyl-CoA carboxylase (PCCA) (c.937C>T/c, 937C>T; pArg313Stop/p.Arg313Stop can result in the loss of the PCCA active site and loss of domains responsible for PCCA interaction with the β subunits of propionyl-CoA carboxylase (PCCB). A non-limiting list of examples of PCCA mutations and PCCB mutations can be found at the following links: http://cbs.lf1.cuni.cz/pcc/list_of_pcca_mutations.htm and http://cbs.lf1.cuni.cz/pcc/list_of_pccb_mutations.htm, respectively.

The inability to convert propionyl-CoA to methylmalonyl-CoA results in the buildup of certain metabolites, some of which are toxic. The sources of propionyl-CoA include valine, isoleucine, threonine, methionine, odd-chain fatty acids, and cholesterol. The resulting impaired metabolism of these metabolites causes a buildup of metabolites that have deleterious effects on various target organs, e.g. heart, central nervous system etc., considerably shortening the lifespan of affected patients and severely limiting their diet and lifestyle.

Methylmalonic acidemia (MMA) is caused by dysfunction of methylmalonyl-CoA mutase (MM-CoA mutase, or MCM), the mitochondrial enzyme that catalyzes the conversion of methylmalonyl-CoA to succinyl-CoA using adenosylcobalamin (AdoCbl) as a cofactor. The conversion can involve two steps. First step is to convert D-methylmalonyl-CoA to L-methylmalonyl-CoA catalyzed by methylmalonyl-CoA racemase. The second step is to convert L-methylmalonyl-CoA to succinyl-CoA catalyzed by methylmalonyl-CoA mutase. MCM is essential in the normal catabolism of branched-chain amino acids such as leucine and valine as well as methionine, threonine, odd-chain fatty acids and cholesterol. The dysfunction of MCM results in accumulation of methylmalonyl-CoA, methylmalonic acid, as well as the same metabolites that build up in PA listed above. The sources of methylmalonyl-CoA can include, but are not limited to valine, leucine, isoleucine, threonine, methionine, odd-chain fatty acids, and cholesterol.

The failure of properly converting propionyl-CoA to methylmalonyl-CoA, or the failure of properly converting methylmalonyl-CoA to succinyl-CoA, results in accumulation of propionyl-CoA and a derived organic acid, 2-methylcitric acid which disrupts normal Krebs cycle function, also called citric acid cycle or tricarboxylic acid (TCA) cycle. In addition, accumulation of propionyl-CoA results in the inhibition of N-acetylglutamate synthase (NAGS) and consequently lower levels of N-acetylglutamate, resulting in inhibition of urea cycle function (decreased conversion of ammonia to urea) which can lead to hyperammonemia. Together, this metabolic dysregulation leads to the signs and symptoms of PA and MMA.

Therefore, therapeutic strategies which reduce the amount of propionyl-CoA, methylmalonyl-CoA, and/or their related metabolites, and combinations thereof, can be used to treat PA, MMA, as well as other metabolic disorders associated with the production of propionyl-CoA and methylmalonyl-CoA. Non-limiting examples of such metabolic disorders, e.g., disorders involving a BCAA pathway, include isovaleric acidemia, mitochondrial short-chain enoyl-CoA hydratase 1 deficiency (OMIM 616277; ECHS₁ deficiency)), 3-hydroxyisobutyryl-CoA hydrolase deficiency (OMIM 250620; HIBCH deficiency), 3-hydroxyisobutyrate dehydrogenase deficiency, methylmalonate-semialdehyde dehydrogenase deficiency (OMIM 614105), 2-methyl-3-hydroxybutyryl-CoA dehydrogenase deficiency (OMIM 300438; HSD₁₀ deficiency), 2-methylacetoacetyl-CoA thiolase deficiency (OMIM 203750, ACAT1 deficiency), 3-methylcrotonyl-CoA carboxylase deficiency (MCCD), and 3-hydroxy-3-methylglutaric aciduria (HMGD).

Isovaleric acidemia (IVA) is a type of organic acid disorder in which affected individuals have problems breaking down leucine, which results in the accumulation of toxic levels of leucine, 2-ketoisocaproic acid (KICA), isovaleryl-CoA and isovaleric acid. IVA is caused by mutations in the IVD gene and is an autosomal recessive metabolic disorder. Signs and symptoms may range from very mild to life-threatening. In severe cases, symptoms begin within a few days of birth and include poor feeding, vomiting, seizures, and lack of energy (lethargy); these may progress to more serious medical problems including seizures, coma, and possibly death. In other cases, signs and symptoms appear during childhood and may come and go over time. A characteristic sign of IVA is a distinctive odor of sweaty feet during acute illness. Other features may include failure to thrive or delayed development.

Mitochondrial short-chain enoyl-CoA hydratase 1 deficiency (ECHS1D; OMIM 616277) is caused by a dysfunction of short-chain enoyl-CoA hydratase (ECHS1; EC 4.2.1.17; formerly called SCEH). ECHS1 is a mitochondrial enzyme that catalyzes the conversion of unsaturated trans-2-enoyl-CoA species to their corresponding 3(S)-hydroxyacyl-CoA species. ECHS1 is essential for the normal catabolism of the branched-chain amino acids, isoleucine and valine, and also functions in the β-oxidation of short- and medium-chain fatty acids. The clinical phenotype of ECHS1 deficiency is not consistent with that of a fatty acid oxidation disorder, suggesting that this is primarily a disorder of branched-chain amino acid metabolism. ECHS1 deficiency is characterized by the accumulation of abnormal metabolites including: S-(2-carboxypropyl)cysteine, S-(2-carboxypropyl)cysteamine, N-acetyl-S-(2-carboxypropyl)cysteine, S-(2-carboxypropyl)cysteine carnitine, methacrylylglycine, S-(2-carboxyethyl)cysteine, S-(2-carboxyethyl)cysteamine, N-acetyl-S-(2-carboxyethyl)cysteine and 2,3-dihydroxy-2-methylbutyric acid. Therefore, therapeutic strategies which reduce the production of the above metabolites can be used to treat mitochondrial short-chain enoyl-CoA hydratase 1 deficiency.

Methylacrylic aciduria (OMIM 250620; also called 3-hydroxyisobutyryl-CoA hydrolase deficiency) is caused by dysfunction of 3-hydroxyisobutyryl-CoA hydrolase (HIBCH; EC 3.1.2.4), the mitochondrial enzyme that catalyzes the conversion of 3-hydroxyisobutyryl-CoA to free 3-hydroxyisobutyrate. HIBCH is essential in the normal catabolism of the branched-chain amino acid valine. HIBCH is also reactive towards 3-hydroxypropionyl-CoA, giving it a dual role in a secondary pathway of propionate metabolism. The sources of hydroxypropionyl-CoA can include, but are not limited to valine, leucine, isoleucine, threonine, methionine, odd-chain fatty acids and cholesterol. HIBCH deficiency results in the accumulation of abnormal metabolites including: (S)-3-hydroxyisobutyryl-L-carnitine, S-(2-carboxypropyl)cysteine, S-(2-carboxypropyl)cysteamine, N-acetyl-S-(2-carboxypropyl)cysteine, S-(2-carboxypropyl)cysteine carnitine, methacrylylglycine, S-(2-carboxyethyl)cysteine, S-(2-carboxyethyl)cysteamine, N-acetyl-S-(2-carboxyethyl)cysteine and 2,3-dihydroxy-2-methylbutyric acid. Therefore, therapeutic strategies which reduce the production of the above metabolites can be used to treat methylacrylic aciduria.

3-hydroxyisobutyrate dehydrogenase (HIBADH; EC1.1.1.31) deficiency may be caused by mutations in the HIBADH gene, encoding an enzyme that catalyzes the NAD(+)-dependent, reversible oxidation of 3-hydroxyisobutyrate to methylmalonate semialdehyde, although no mutations have been identified as causing this disease. 3-hydroxyisobutyrate dehydrogenase deficiency may also be caused by defects in respiratory chain function such as Leigh's syndrome. HIBADH is essential in the normal catabolism of the branched-chain amino acid valine. HIBADH deficiency is one cause of 3-hydroxyisobutyric aciduria, a disorder with a heterogeneous clinical phenotype that can also be caused by defects in the electron transport chain or by methylmalonate semialdehyde dehydrogenase deficiency. The dysfunction of HIBADH has been shown to result in accumulation of 3-hydroxyisobutyrate and 3-hydroxyisobutyryl carnitine. Therefore, therapeutic strategies which reduce production of the above metabolites can be used to treat 3-hydroxyisobutyrate dehydrogenase deficiency.

Methylmalonate semialdehyde dehydrogenase deficiency (MMSDHD; OMIM 614105) is caused by the deficiency of the enzyme methylmalonate semialdehyde dehydrogenase (MMSDH; EC 1.2.1.27). MMSDH is encoded by the ALDH6A1 gene and catalyzes the oxidative decarboxylation of methylmalonate semialdehyde into propionyl-CoA. MMSDH is essential in the normal catabolism of the branched-chain amino acid valine and thymine metabolism. MMSDH deficiency is one cause of 3-hydroxyisobutyric aciduria, a disorder with a heterogeneous clinical phenotype that can also be caused by defects in the electron transport chain or by 3-hydroxyisobutyrate dehydrogenase (HIBADH) deficiency. The dysfunction of MMSDH has been shown to result in accumulation of 3-hydroxyisobutyrate and 3-hydroxyisobutyryl carnitine, as well as 3-hydroxypropionic acid and 2-ethyl-3-hydroxypropionic acid. Therefore, therapeutic strategies which reduce production of the above metabolites can be used to treat methylmalonate semialdehyde dehydrogenase deficiency.

17-β hydroxysteroid dehydrogenase X deficiency (OMIM 300438) is caused by the deficiency of hydroxysteroid 17-β dehydrogenase 10 (EC 1.1.1.178; also known as 2-methyl-3-hydroxybutyryl-CoA dehydrogenase or 3-hydroxyacyl-CoA dehydrogenase type II). Hydroxysteroid 17-β dehydrogenase 10 (HSD10) is a multifunctional mitochondrial enzyme that catalyzes the reversible conversion of 2-methyl-3-hydroxybutyryl-CoA to 2-methylacetoacetyl-CoA and is an essential enzyme in the degradation pathway of isoleucine. HSD10 is encoded by the gene HSD17B10 (formerly known as HADH2) and HSD10 deficiency is caused by mutations in the HSD17B10 gene. This syndrome has a biochemical phenotype similar to that of β-ketothiolase deficiency, but represents a unique disorder which typically shows a more severe clinical phenotype. HSD10 is known to catalyze the oxidation of a wide variety of steroid receptor modulators and thus plays a role in sex steroid and neuroactive steroid metabolism, and is also a subunit of mitochondrial ribonuclease P which is involved in tRNA maturation. The dysfunction of HSD10 in isoleucine degradation has been shown to result in the accumulation of tiglylglycine, 2-methyl-3-hydroxybutyrate, OH—C5 carnitine, and in some cases 2-ethylhydracrylic acid, 3-hydroxyisobutyrate and tiglylglutamic acid. Therefore, therapeutic strategies which reduce production of the above metabolites can be used to treat 17-β hydroxysteroid dehydrogenase X deficiency.

Alpha-methylacetoacetic aciduria (OMIM 203750) is caused by the deficiency of 3-methylacetoacetyl-CoA thiolase (EC 2.3.1.9; more commonly called β-ketothiolase or T2). β-ketothiolase (β-KT) is a K⁺-dependent mitochondrial enzyme that catalyzes the thiolytic cleavage of 2-methylacetoacetyl-CoA to produce acetyl-CoA and propionyl-CoA. β-KT is an essential enzyme in the degradation pathway of isoleucine. β-KT is encoded by the gene ACAT1 and β-KT deficiency is caused by mutations in the ACAT1 gene. This syndrome has a biochemical phenotype similar to that of HSD10 deficiency, but represents a unique disorder as blockade of isoleucine degradation by loss of R-KT does not commonly cause developmental disabilities except for a few cases with neurological sequelae attributed to severe ketoacidotic attacks. The dysfunction of β-KT in isoleucine degradation has been shown to result in the accumulation of ketones such as 3-hydroxybutyrate, acetoacetic acid, 2-methylacetoacetic acid and 2-butanone, as well as tiglylglycine, 2-methyl-3-hydroxybutyrate, OH—C5 carnitine, and in some cases 2-ethylhydracrylic acid, 3-hydroxyisobutyrate and tiglylglutamic acid. Therefore, therapeutic strategies which reduce production of the above metabolites can be used to treat alpha-methylacetoacetic aciduria.

Other non-limiting examples of CoA disorders that can be treated by the presently disclosed methods include glutaric aciduria type 1, long-chain acyl-CoA dehydrogenase deficiency (LCHAD), very-long chain acyl-CoA dehydrogenase deficiency (VLCAD), and Refsum Disease and the diseases in Table 1.

TABLE 1
Additional Dseases for Treatment by the Disclosed Methods
Disease Name

| | | |
|---|---|---|
| Acromicric dysplasia | Barth syndrome | cytochrome P450 family 17 |
| | | subfamily A member 1 |
| aminoacylase 1 | Inherited thyroxine-binding | acyl-CoA dehydrogenase very |
| | globulin deficiency | long chain |
| Caffey disease | glycyl-tRNA synthetase | L-2-hydroxyglutarate |
| | | dehydrogenase |
| carbonic anhydrase 5A | acyl-CoA dehydrogenase | acyl-CoA oxidase 1 |
| | short/branched chain | |
| collagen type IX alpha 1 | AU RNA binding | metabolism of cobalamin |
| chain | methylglutaconyl-CoA | associated C |
| | hydratase | |
| collagen type IX alpha 3 | hydroxyacyl-CoA | Aminoacylase 1 deficiency |
| chain | dehydrogenase trifunctional | |
| | multienzyme complex subunit | |
| | alpha | |
| hydroxyacyl-CoA | 3-oxoacid CoA-transferase 1 | Short/branched chain acyl- |
| dehydrogenase trifunctional | | CoA dehydrogenase deficiency |
| multienzyme complex | | |
| subunit beta | | |
| Cowden syndrome | platelet activating factor | Dilated cardiomyopathy with |
| | acetylhydrolase 1b regulatory | ataxia syndrome |
| | subunit 1 | |
| Myoclonus-dystonia | hydroxyacyl-CoA | heparan-alpha-glucosaminide |
| | dehydrogenase | N-acetyltransferase |
| retinoic acid receptor alpha | peroxisomal biogenesis factor | fatty acid 2-hydroxylase |
| | 7 | |
| triosephosphate isomerase 1 | hydroxysteroid 17-beta | ACAD9 deficiency |
| | dehydrogenase 4 | |
| Medium-chain acyl-CoA | phytanoyl-CoA 2-hydroxylase | Isobutyryl-CoA dehydrogenase |
| dehydrogenase deficiency | | deficiency |
| Short-chain acyl-CoA | Desmosterolosis | hepatic and glial cell adhesion |
| dehydrogenase deficiency | | molecule |
| Very long-chain acyl-CoA | TGFB induced factor | metabolism of cobalamin |
| dehydrogenase deficiency | homeobox 1 | associated D |
| Cytochrome P450 | ATP binding cassette | Fatty acid hydroxylase- |
| oxidoreductase deficiency | subfamily D member 4 | associated neurodegeneration |
| 3-beta-hydroxysteroid | cytochrome P450 family 7 | LMBR1 domain containing 1 |
| dehydrogenase deficiency | subfamily B member 1 | |
| 17 alpha-hydroxylase/17,20- | Ohdo syndrome, Say-Barber- | tyrosine aminotransferase |
| lyase deficiency | Biesecker-Young-Simpson | |
| | variant | |
| Beta-ketothiolase deficiency | arachidonate 12-lipoxygenase, | GABA-transaminase |
| | 12R type | deficiency |
| Cerebrotendinous | Intervertebral disc disease | 3-hydroxy-3-methylglutaryl- |
| xanthomatosis | | CoA lyase |
| DOORS syndrome | alo-keto reductase family 1 | acyl-CoA synthetase family |
| | member D1 | member 3 |
| Dopamine beta-hydroxylase | acyl-CoA dehydrogenase | Combined malonic and |
| deficiency | family member 8 | methylmalonic aciduria |
| 3-hydroxyacyl-CoA | histone deacetylase 4 | Alpha-methylacyl-CoA |
| dehydrogenase deficiency | | racemase deficiency |
| Glutaric acidemia type I | serine palmitoyltransferase | MEGDEL syndrome |
| | long chain base subunit 1 | |
| Glutaric acidemia type II | pyruvate dehydrogenase | CLPB deficiency |
| | phosphatase catalytic subunit | |
| | 1 | |
| propionyl-CoA carboxylase | Propionic acidemia | Multiple epiphyseal dysplasia |
| subunit alpha | | |
| propionyl-CoA carboxylase | Genitopatellar syndrome | collagen type IX alpha 2 chain |
| subunit beta | | |
| Glycogen storage disease | solute carrier family 25 | Hereditary multiple |
| type V | member 19 | osteochondromas |
| Congenital bile acid | cytochrome P450 family 27 | lysine acetyltransferase 6B |
| synthesis defect type 2 | subfamily A member 1 | |
| aminomethyltransferase | malonyl-CoA decarboxylase | Stickler syndrome |
| dihydrolipoamide | acyl-CoA dehydrogenase | Saethre-Chotzen syndrome |
| dehydrogenase | short chain | |
| Isovaleric acidemia | acyl-CoA dehydrogenase | Corticosterone methyloxidase |
| | medium chain | deficiency |
| Succinyl-CoA:3-ketoacid | isovaleryl-CoA | 3-methylcrotonyl-CoA |
| CoA transferase deficiency | dehydrogenase | carboxylase deficiency |
| 3-hydroxy-3-methylglutaryl- | Amish lethal microcephaly | pyruvate dehydrogenase E1 |
| CoA lyase deficiency | | beta subunit |
| Dihydrolipoamide | arachidonate lipoxygenase 3 | Tyrosinemia |
| dehydrogenase deficiency | | |
| Malonyl-CoA decarboxylase | metabolism of cobalamin | Methylmalonic acidemia with |
| deficiency | associated A | homocystinuria |
| 3-methylglutaconyl-CoA | metabolism of cobalamin | Methylmalonic acidemia |
| hydratase deficiency | associated B | |
| Mitochondrial complex I | acetyl-CoA acetyltransferase 1 | hydroxysteroid 17-beta |
| deficiency | | dehydrogenase 10 |
| Costeff syndrome | methylmalonyl-CoA | pyruvate dehydrogenase E1 |
| | epimerase | alpha 1 subunit |
| D-bifunctional protein | pyruyate dehydrogenase | Pyruvate dehydrogenase |
| deficiency | complex component X | deficiency |
| Peroxisomal acyl-CoA | dihydrolipoamide S- | Distal hereditary motor |
| oxidase deficiency | acetyltransferase | neuropathy, type V |
| Succinic semialdehyde | glutaryl-CoA dehydrogenase | Auriculo-condylar syndrome |
| dehydrogenase deficiency | | |
| host cell factor C1 | methylcrotonoyl-CoA | Megalencephalic |
| | carboxylase 1 | leukoencephalopathy with |
| | | subcortical cysts |
| EBP, cholestenol delta- | methylcrotonoyl-CoA | alpha-methylacyl-CoA |
| isomerase | carboxylase 2 | racemase |
| myotubularin 1 | Long-chain 3-hydroxyacyl- | 4-hydroxyphenylpyruvate |
| | CoA dehydrogenase | dioxygenase |
| | deficiency | |
| HSD 1 0 disease | biotinidase | 2-hydroxyglutaric aciduria |
| X-linked sideroblastic | methylmalonyl-CoA mutase | |
| anemia | | |

The present disclosure provides methods of treating metabolic disorders (e.g., organic acidemias) by reducing the formation of metabolites associated with such metabolic disorders. In some embodiments, the present disclosure provides methods of treating organic acidemia comprising reducing the formation and/or amount of metabolites associated with organic acidemia. In some embodiments, the methods described herein can be used to treat any disease or disorder associated with the metabolism of branched-chain amino acids. In particular embodiments, the present disclosure provides for methods of reducing isovaleryl-CoA, propionyl-CoA and/or methylmalonyl-CoA production in a subject. In some embodiments, the present disclosure provides methods for treating IVA, PA, and MMA, thereby addressing key needs in the fields of metabolic disorder therapeutics.

In some embodiments, the level of a metabolite that is associated with organic acidemia patients (e.g., isovaleryl-CoA, propionyl-CoA or methylmalonyl-CoA), is reduced by at least about 1% to about 100%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, inclusive of all values and subranges therebetween, compared to its counterpart without the treatment of the inhibitor. For example, the reduced level may be at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%.

Disclosed herein, in various embodiments, are methods and compositions for treating organic acidemias comprising administering compounds that are capable of forming a coenzyme A (CoA) ester or a carnitine ester. In some embodiments, such compounds comprise a carboxylic acid (or similar group having a carbonyl or imine and a leaving group) attached to a pharmaceutically acceptable core. Non-limiting examples of similar groups include carboxylic acid esters (RCO₂R′, wherein R and R′ are e.g., alkyl, aryl, activated esters, etc.), thioesters (RC(O)SR′), amides (RC(O)NR′R″, e.g., primary, secondary, tertiary, and Weinreb amides), acid chlorides (RCOX, X=halogen), acid anhydrides (RC(O)OC(O)R′), acyl sulfonates (RC(O)OS(O)₂OR′, acyl phosphates (RC(O)OP(O)OR′₂), or carboxyimidates (R(C═NR″)OR′). In some embodiments, the pharmaceutically acceptable core does not include an electron withdrawing group. In some embodiments, the core is substituted at the alpha position to the carboxylic acid (or similar group). In some embodiments, the pharmaceutically acceptable core comprises a saturated or unsaturated hydrocarbon region, which may be linear, branched, or cyclic (including carbocyclyl and heterocyclyl groups), and can be optionally substituted. Non limiting examples of such hydrocarbons include alkyl, alkenyl, alkynyl, cycloalkyl, aryl, and heteroaryl groups. In some embodiments, the hydrocarbon region comprises one or more heteroatoms. In some embodiments, the pharmaceutically acceptable core has a molecular weight of less than or equal to about 2000 Da, less than or equal to about 1000 Da, or less than or equal to about 500 Da, e.g., about 450, about 400, about 350, about 300, about 250, about 200, about 150, about 100 or less, inclusive of all values and subranges therebetween.

In some embodiments, the compounds suitable for the methods disclosed herein are represented by Formula (I):

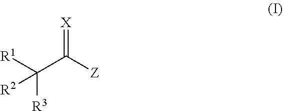

or a CoA ester or carnitine ester thereof, or a pharmaceutically acceptable salt, solvate, or ester thereof,

wherein:
X is O, NH, or S;
Z is OR⁴, NR⁴R⁴, SR⁴, halide, or leaving group;
each of R¹, R² and R³ are independently H, halide, alkyl, alkenyl, alkynyl, carbocyclyl, carbocyclylalkyl, heterocyclyl, or heterocyclylalkyl, provided that at least one of R¹, R² and R³ is not H;
or any two of R¹, R² and R³ may be taken together with the carbon atom to which they are attached to form a carbocyclyl or heterocyclyl;
each R⁴ is independently H, alkyl, haloalkyl, alkenyl, alkynyl, carbocyclyl, carbocyclylalkyl, heterocyclyl, heterocyclylalkyl, —C(O)R⁵, —SO₂R⁵, —P(O)(OR⁵)₂, or

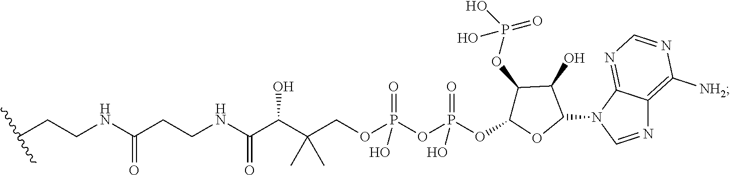

R⁵ is alkyl, haloalkyl, alkenyl, alkynyl, carbocyclyl, carbocyclylalkyl, heterocyclyl, heterocyclylalkyl, or arylalkyl;
wherein each hydrogen is independently optionally replaced with a halide or a deuterium, and
wherein administration of the compound reduces at least one metabolite that accumulates in an organic acidemia patient.

In some embodiments of Formula (I), X is O, NH, or S; Z is OR⁴, NR⁴R⁴, or SR⁴; each of R¹, R² and R³ are independently H, alkyl, alkenyl, alkynyl, carbocyclyl, carbocyclylalkyl, heterocyclyl, or heterocyclylalkyl, provided that at least one of R¹, R² and R³ is not H; or any two of R¹, R² and R³ may betaken together with the carbon atom to which they are attached to form a carbocyclyl or heterocyclyl; each R⁴ is independently H, alkyl, haloalkyl, alkenyl, alkynyl, carbocyclyl, carbocyclylalkyl, heterocyclyl, heterocyclylalkyl, —C(O)R⁵, —SO₂R⁵, or —P(O)(OR⁵)₂-R⁵ is alkyl, haloalkyl, alkenyl, alkynyl, carbocyclyl, carbocyclylalkyl, heterocyclyl, heterocyclylalkyl, or arylalkyl; wherein each hydrogen is independently optionally replaced with a halide or a deuterium, and wherein administration of the compound reduces at least one metabolite that accumulates in an organic acidemia patient.

In some embodiments, the compound of Formula (I) is a CoA thioester, wherein Z is SR⁴, and R⁴ is:

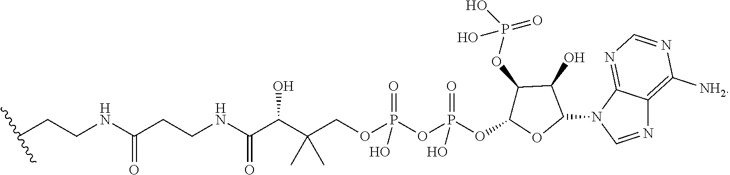

As known in the art, Coenzyme A is [[(2R,3S,4R,5R)-5-(6-aminopurin-9-yl)-4-hydroxy-3-phosphonooxyoxolan-2-yl]methoxy-hydroxyphosphoryl] [(3R)-3-hydroxy-2,2-dimethyl-4-oxo-4-[[3-oxo-3-(2-sulfanylethylamino)propyl]amino]butyl] hydrogen phosphate. An example of a CoA ester of 2,2-dimethylbutyric acid is provided herein.

In some embodiments, X is O. In other embodiments, X is S. In yet another embodiment, X is NH.

In some embodiments, Z is OR⁴, NR⁴R⁴, SR⁴. In certain embodiments, Z is OR⁴. In other embodiments, Z is a leaving group. The leaving group, as defined herein, can be any suitable leaving group known in the art. In some embodiments, each R⁴ is independently H, alkyl, carbocyclyl or carbocyclylalkyl. In some embodiments, each R⁴ is independently H or alkyl. In some embodiments, the alkyl is a C₁₋₄ alkyl. In some embodiments, the C₁₋₄ alkyl is selected from the group consisting of methyl, ethyl, n-propyl, n-butyl, or t-butyl. In some embodiments, R⁴ is H. In some embodiments, the carbocyclyl is a C₃₋₆ carbocyclyl. In some embodiments, the carbocyclyl is cyclopropane.

In some embodiments, each of R¹, R² and R³ are independently H, halogen, alkyl, alkenyl, alkynyl, carbocyclyl, carbocyclylalkyl, heterocyclyl, or arylalkyl. In certain embodiments, the alkyl is a C₁₋₆ alkyl, the alkenyl is a C₂₋₆ alkenyl, the alkynyl is a C₂₋₆ alkynyl, the carbocyclyl is a C₃₋₁₂ cycloalkyl or a C₆₋₁₂ aryl, and the heterocyclyl is a C₃₋₁₂ heterocyclyl.

In some embodiments, each of R¹, R² and R³ is alkyl. In other embodiments, two of R¹, R² and R³ are alkyl. In certain embodiments, two of R¹, R² and R³ are alkyl, wherein the remaining R¹, R² and R³ is H. In still other embodiments, one of R¹, R² and R³ are alkyl. In some embodiments, the alkyl is a C₁₋₆ alkyl. In some embodiments, R² is not propyl. In some embodiments, R³ is not propyl. In certain embodiments, when R¹ is H, X is O and Z is OH, each of R² and R³ are not propyl, i.e., the compound is not valproic acid having the structure

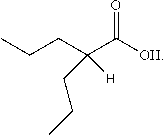

In some embodiments, any two of R¹, R² and R³ taken together with the carbon atom to which they are attached forms a carbocyclyl or heterocyclyl. In some embodiments, any two of R¹, R² and R³ taken together with the carbon atom to which they are attached forms a carbocyclyl or heterocyclyl, wherein the remaining R¹, R² and R³ is H or alkyl. In certain embodiments, the carbocyclyl is a C₃₋₁₂ cycloalkyl or a C₆₋₁₂ aryl, and the heterocyclyl is a C₃₋₁₂ heterocyclyl. In certain other embodiments, the alkyl is a C₁₋₆ alkyl.

In some embodiments, the compounds suitable for the methods described herein are represented by Formula (IA):

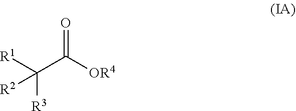

or a CoA ester or carnitine ester thereof, or a pharmaceutically acceptable salt, solvate, or ester thereof,

wherein:

each of R¹, R² and R³ is independently H, alkyl, or carbocyclyl, provided that at least one of R¹, R² and R³ is not H; and

R⁴ is H or alkyl.

In some embodiments, each of R¹, R² and R³ is independently H or alkyl, provided that at least one of R¹, R² and R³ is not H. In some embodiments, each of R¹, R² and R³ is independently H, alkyl, or carbocyclyl, provided that at least two of R¹, R² and R³ is not H. In some embodiments, each of R¹, R² and R³ is independently H or alkyl, provided that at least two of R¹, R² and R³ is not H.

In some embodiments, at least one of R¹, R² and R³ is alkyl. In some embodiments, at least two of R¹, R² and R³ are alkyl. In some embodiments, each of R¹, R² and R³ is alkyl. In some embodiments, R¹ and R² are alkyl, and R³ is H. In some embodiments, R¹ and R² are H, and R³ is alkyl. In some embodiments, R¹ and R² are H, and R³ is alkyl. In some embodiments, R¹ and R² are H, and R³ is carbocyclyl. In some embodiments, R¹ and R² are alkyl and R³ is carbocyclyl. In some embodiments, the alkyl is a C₁₋₄ alkyl. In some embodiments, the C₁₋₄ alkyl is selected from the group consisting of methyl, ethyl, n-propyl, n-butyl, or 1-butyl. In some embodiments, the alkyl is methyl. In some embodiments, the alkyl is ethyl. In some embodiments, the alkyl is butyl. In some embodiments, the carbocyclyl is cyclopropyl. In some embodiments, R¹ and R² are methyl and R³ is methyl, ethyl, n-propyl, n-butyl, or t-butyl. In some embodiments, R¹ and R² are methyl and R³ is ethyl.

In some embodiments, R⁴ is alkyl. In some embodiments, the alkyl is a C₁₋₄ alkyl. In some embodiments, the C₁₋₄ alkyl is selected from the group consisting of methyl, ethyl, n-propyl, n-butyl, or t-butyl. In some embodiments, R⁴ is H.

In certain embodiments, when R¹ is H, X is O and Z is OH, each of R² and R³ are not propyl, i.e., the compound is not valproic acid having the structure

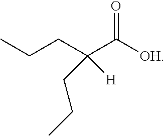

In some embodiments, the compounds suitable for the methods described herein are represented by Formula (II):

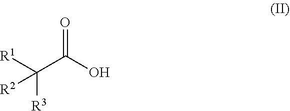

or a CoA ester or carnitine ester thereof, or a pharmaceutically acceptable salt, solvate, or ester thereof,

wherein:
each of R¹, R² and R³ is independently H, halogen, alkyl, alkenyl, alkynyl, carbocyclyl, carbocyclylalkyl, heterocyclyl, or heterocyclylalkyl, provided that at least one of R¹, R² and R³ is not H;
or any two of R¹, R² and R³ taken together with the carbon atom to which they are attached forms a carbocyclyl or heterocyclyl.

In some embodiments, when each of R¹, R² and R³, are independently H, halogen, alkyl, alkenyl, alkynyl, carbocyclyl, carbocyclylalkyl, heterocyclyl, or arylalkyl, provided that at least one of R¹, R² and R³ is H. In certain embodiments, the alkyl is a C₁₋₆ alkyl, the alkenyl is a C₂₋₆ alkenyl, the alkynyl is a C₂₋₆ alkynyl, the carbocyclyl is a C₃₋₁₂ cycloalkyl or a C₆₋₁₂ aryl, and the heterocyclyl is a C₃₋₁₂ heterocyclyl.

In some embodiments, each of R¹, R² and R³ is alkyl. In some embodiments, at least two of R₁, R₂ and R₃ are alkyl. In other embodiments, two of R¹, R² and R³ are alkyl. In certain embodiments, two of R¹, R² and R³ are alkyl, wherein the remaining R¹, R² and R³ is H. In still other embodiments, one of R¹, R² and R³ are alkyl. In some embodiments, the alkyl is a C₁₋₆ alkyl. In some embodiments, R² is not propyl. In some embodiments, R³ is not propyl. In certain embodiments, when R¹ is H, each of R₂ and R₃ are not propyl, i.e., the compound is not valproic acid having the structure

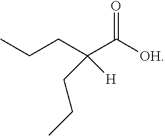

In some embodiments, any two of R¹, R² and R³ taken together with the carbon atom to which they are attached forms a carbocyclyl, or heterocyclyl, wherein the remaining R¹, R² and R³ is H, halogen, alkyl, alkenyl, alkynyl, carbocyclyl, carbocyclylalkyl (e.g., or arylalkyl), heterocyclyl, or heterocyclylalkyl. In certain embodiments, the alkyl is a C₁₋₆ alkyl, the alkenyl is a C₂₋₆ alkenyl, the alkynyl is a C₂₋₆ alkynyl, the carbocyclyl is a C₃₋₁₂ cycloalkyl or a C₆₋₁₂ aryl, and the heterocyclyl is a C₃₋₁₂ heterocyclyl. When two of R¹, R² and R³ taken are together to form an aromatic ring (e.g., aryl or heteroaryl), one of R¹, R² and R³ is absent. In some embodiments, the carbocyclyl is not a benzyl substituted at the 3 position with a 1,2,4-oxadiazole.

In some embodiments, any two of R¹, R² and R³ taken together with the carbon atom to which they are attached forms a carbocyclyl or heterocyclyl, wherein the remaining R¹, R² and R³ is H or alkyl, carbocyclylalkyl or heterocyclylalkyl. In certain embodiments, the alkyl is a C₁₋₆ alkyl, the carbocyclyl is a C₃₋₁₂ cycloalkyl or a C₆₋₁₂ aryl, and the heterocyclyl is a C₃₋₁₂ heterocyclyl.

In some embodiments, the pharmaceutically acceptable salt of Formula I, IA, or II suitable for the methods disclosed herein is a sodium salt, a magnesium salt, a calcium salt, a zinc salt, a potassium salt, or a tris(hydroxymethyl)aminomethane salt. In some embodiments, the pharmaceutically acceptable salt is a sodium salt.

In some embodiments, the compound of Formula I, IA, or II suitable for the methods described herein is:

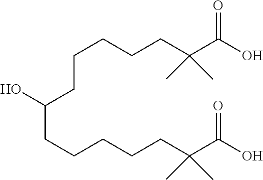 [SMILES: CC(C)(CCCCCC(O)CCCCCC(C)(C)C(=O)O)C(=O)O]

(bempedoic acid) or

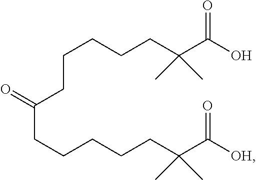 [SMILES: CC(C)(CCCCCC(=O)CCCCCC(C)(C)C(=O)O)C(=O)O]

or a pharmaceutically acceptable salt, solvate, or ester thereof, including CoA derivatives thereof.

In some embodiments, the compounds suitable for the methods described herein are represented by Formula (IIA):

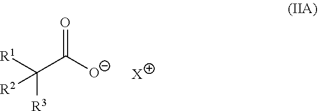

or a pharmaceutically acceptable solvate thereof,

wherein:
each of R¹, R² and R³ is independently H, alkyl, or carbocyclyl, provided that at least one of R¹, R² and R³ is not H; and
X is Na, 1/2Mg, 1/2Ca, 1/2Zn, K, or C(CH₂OH)₃NH₄.

In some embodiments, X is Na.

In some embodiments, each of R¹, R² and R³ is independently H or alkyl, provided that at least one of R¹, R² and R³ is not H. In some embodiments, each of R¹, R² and R³ is independently H, alkyl, or carbocyclyl, provided that at least two of R¹, R² and R³ is not H. In some embodiments, each of R¹, R² and R³ is independently H or alkyl, provided that at least two of R¹, R² and R³ is not H.

In some embodiments, at least one of R¹, R² and R³ is alkyl. In some embodiments, at least two of R¹, R² and R³ are alkyl. In some embodiments, each of R¹, R² and R³ is alkyl. In some embodiments, R¹ and R² are alkyl, and R³ is H. In some embodiments, R¹ and R² are H, and R³ is alkyl. In some embodiments, R and R² are H, and R³ is alkyl. In some embodiments, R¹ and R² are H, and R³ is carbocyclyl. In some embodiments, the alkyl is a C₁₋₄ alkyl. In some embodiments, the C₁₋₄ alkyl is selected from the group consisting of methyl, ethyl, n-propyl, n-butyl, or t-butyl. In some embodiments, the carbocyclyl is cyclopropyl.

Non-limiting examples of compounds that fall within Formula I, IA, and II are provided in Tables 2A and 2B:

TABLE 2A
Carboxylic Acid Compounds of Formula I, IA, and II.

| Compound No. | Structure | Name |
|---|---|---|
| Compound 1 | 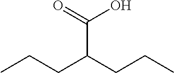 | 2-propylpentanoic acid (valproic acid) |
| Compound 2 | 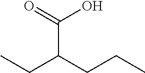 | 2-ethylpentanoic acid |
| Compound 3 | 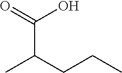 | 2-methylpentanoic acid |
| Compound 4 | 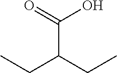 | 2-ethylbutanoic acid |
| Compound 5 | 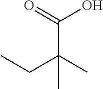 | 2,2-dimethybutanoic acid |
| Compound 6 | 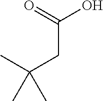 | 3,3-dimethylbutanoic acid |
| Compound 7 | 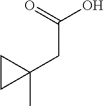 | 2-(1- methylcyclopropyl) acetic acid |
| Compound 8 | 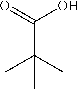 | 2,2,dimethylpropionic acid |
| Compound 9 | 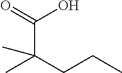 | 2,2-dimethylpentanoic acid |
| Compound 10 | 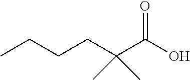 | 2,2-dimethylhexanoic acid |
| Compound 11 | 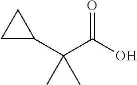 | 2-cyclopropyl- methylpropanoic acid |
| Compound 12 | 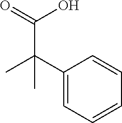 | 2-methyl-2- phenylpropanoic acid |
| Compound 13 | 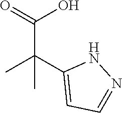 | 2-methyl-2-(1H-pyrazol- 5-yl)propanoic acid |
| Compound 14 | 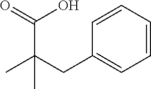 | 2,2-dimethyl-3- phenylpropanoic acid |
| Compound 15 | 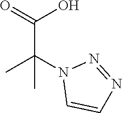 | 2-methyl-2-(1H-1,2,3- triazol-1-yl)propanoic acid |

TABLE 2B
Ester Compounds of Formula I, IA, and II

| Compound No. | Structure | R⁴ | | |
|---|---|---|---|---|
| Compound 16 | 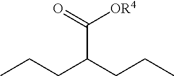 | Me (16A) | Et (16B) | t-Bu (16C) |
| Compound 17 | 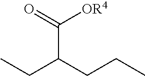 | Me (17A) | Et (17B) | t-Bu (17C) |
| Compound 18 | 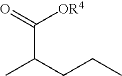 | Me (18A) | Et (18B) | t-Bu (18C) |
| Compound 19 | 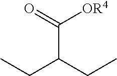 | Me (19A) | Et (19B) | t-Bu (19C) |
| Compound 20 | 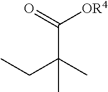 | Me (20A) | Et (20B) | t-Bu (20C) |
| Compound 21 | 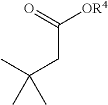 | Me (21A) | Et (21B) | t-Bu (21C) |
| Compound 22 | 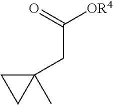 | Me (22A) | Et (22B) | t-Bu (22C) |
| Compound 23 | 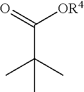 | Me (23A) | Et (23B) | t-Bu (23C) |
| Compound 24 | 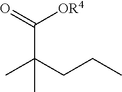 | Me (24A) | Et (24B) | t-Bu (24C) |
| Compound 25 | 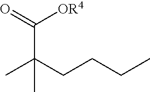 | Me (25A) | Et (25B) | t-Bu (25C) |
| Compound 26 | 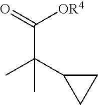 | Me (26A) | Et (26B) | t-Bu (26C) |
| Compound 27 | 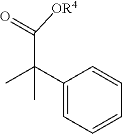 | Me (27A) | Et (27B) | t-Bu (27C) |
| Compound 28 | 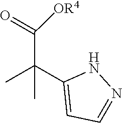 | Me (28A) | Et (28B) | t-Bu (28C) |
| Compound 29 | 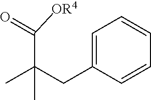 | Me (29A) | Et (29B) | t-Bu (29C) |
| Compound 30 | 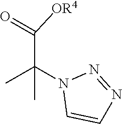 | Me (30A) | Et (30B) | t-Bu (30C) |

In some embodiments, the compound of Formula I, IA, or II is 2,2-dimethylbutyric acid. 2,2-dimethylbutyric acid is represented by the structure (5).

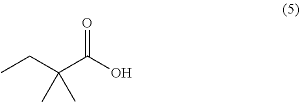

(5) 2,2-dimethylbutyric acid (also known as 2,2-dimethylbutanoic acid or 2,2-dimethylbutyrate; CAS No. 595-37-9).

In some embodiments, the present disclosure provides a method of treating a patient with 2,2-dimethylbutyric acid or pharmaceutically acceptable salt thereof that is biotransformed into 2,2-dimethylbutyryl-CoA in vivo. In some embodiments, the method comprises treating a patient with a compound 2,2-dimethylbutyric acid or pharmaceutically acceptable salt thereof that forms 2,2-dimethylbutyryl-CoA in an intracellular compartment

Without being bound by theory, the compounds of the present disclosure can be administered as a free acid or a pharmaceutically acceptable salt, and the compound can be converted (i.e., metabolized) in vivo to form one or more therapeutically active metabolites that effectively treat the diseases disclosed herein, e.g., PA and MMA. In some embodiments, the metabolites of 2,2-dimethylbutyric acid suitable for use in the disclosed methods include 2,2-dimethylbutyryl-CoA and 2,2-dimethylbutyryl-carnitine.

The structure of 2,2-dimethylbutyryl-carnitine is provided below:

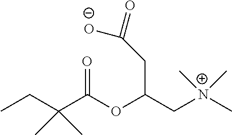 [SMILES: CCC(C)(C)C(=O)OC(CC(=O)[O-])C[N+](C)(C)C]

In some embodiments, the 2,2-dimethylbutyryl-carnitine is 2,2-dimethylbutyryl-L-carnitine having the structure:

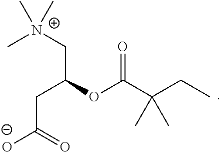 [SMILES: CCC(C)(C)C(=O)O[C@@H](CC(=O)[O-])C[N+](C)(C)C]

The structure of 2,2-dimethylbutyryl-CoA is provided below:

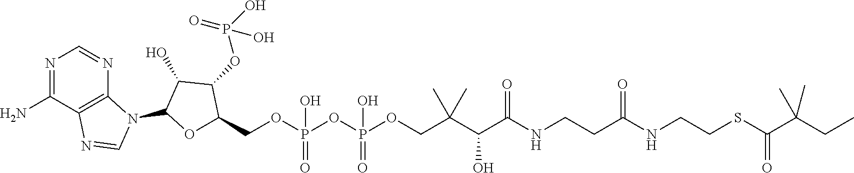

Such compounds of Formula I, Formula IA, Formula II, and Formula IIA reduce at least one metabolite that would otherwise accumulate in an organic acidemia patient (e.g., by about 1-100%, including all values and ranges therebetween), thereby treating the organic acidemia levels. In some embodiments, the at least one metabolite comprises 2-ketoisocaproate, isovaleryl-CoA, 3-methylcrotonyl-CoA, 3-methylglutaconyl-CoA, 3-OH-3-methylglutaryl-CoA, 2-keto-3-methylvalerate, 2-methylbutyryl-CoA, tiglyl-CoA, 2-methyl-3-OH-butyryl-CoA, 2-methyl-acetoacetyl-CoA, 2-ketoisovalerate, isobutyryl-CoA, methylacrylyl-CoA, 3-OH-isobutyryl-CoA, 3-OH-isobutyrate, methylmalonic semialdehyde, propionyl-CoA, or methylmalonyl-CoA, or combinations thereof. In other embodiments, the at least one metabolite comprises propionic acid, 3-hydroxypropionic acid, methylcitrate, glycine, or propionylcarnitine, or combinations thereof.

In some embodiments, the methods disclosed herein can be used to treat PA. In other embodiments, the methods disclosed herein can be used to treat MMA. In still other embodiments, the methods disclosed herein can be used to treat IVA.

In some embodiments, the compounds of Formula I, Formula IA, Formula II, and Formula IIA, when administering to a subject in need thereof, will provide a mean plasma concentration profile within the range of 1 ng/mL to about 500 mg/mL, e.g., about 1 ng/mL, about 10 ng/mL, 20 ng/mL, about 30 ng/mL, about 40 ng/mL, about 50 ng/mL, about 60 ng/mL, about 70 ng/mL, about 80 ng/mL, about 90 ng/mL, about 100 ng/mL, about 110 ng/mL, about 120 ng/mL, about 130 ng/mL, about 140 ng/mL, about 150 ng/mL, about 200 ng/mL, about 300 ng/mL, about 400 ng/mL, about 500 ng/mL, about 600 ng/mL, about 700 ng/mL, about 800 ng/mL, about 900 ng/mL, about 1000 ng/mL, about 1100 ng/mL, about 1200 ng/mL, about 1300 ng/mL, about 1400 ng/mL, about 1500 ng/mL, about 1600 ng/mL, about 1700 ng/mL, about 1800 ng/mL, about 1900 ng/mL, about 2000 ng/mL, about 3100 ng/mL, about 3200 ng/mL, about 3300 ng/mL, about 3400 ng/mL, about 3500 ng/mL, about 3600 ng/mL, about 3700 ng/mL, about 3800 ng/mL, about 3900 ng/mL, about 4000 ng/mL, about 5000 ng/mL, about 6000 ng/mL, about 7000 ng/mL, about 8000 ng/mL, about 9000 ng/mL, about 10000 ng/mL, about 20000 ng/mL, about 30000 ng/mL, about 40000 ng/mL, about 50000 ng/mL, about 60000 ng/mL, about 70000 ng/mL, about 80000 ng/mL, about 90000 ng/mL, about 100000 ng/mL, about 100000 ng/mL, about 200000 ng/mL, about 300000 ng/mL, about 400000 ng/mL, about 500000 ng/mL, about 600000 ng/mL, about 700000 ng/mL, about 800000 ng/mL, about 900000 ng/mL, about 1 mg/mL, about 10 mg/mL, 20 mg/mL, about 30 mg/mL, about 40 mg/mL, about 50 mg/mL, about 60 mg/mL, about 70 mg/mL, about 80 mg/mL, about 90 mg/mL, about 100 mg/mL, about 110 mg/mL, about 120 mg/mL, about 130 mg/mL, about 140 mg/mL, about 150 mg/mL, about 160 mg/mL, about 170 mg/mL, about 180 mg/mL, about 190 mg/mL, about 200 mg/mL, about 210 mg/mL, about 220 mg/mL, about 230 mg/mL, about 240 mg/mL, about 250 mg/mL, about 260 mg/mL, about 270 mg/mL, about 280 mg/mL, about 290 mg/mL, about 300 mg/mL, about 310 mg/mL, about 320 mg/mL, about 330 mg/mL, about 340 mg/mL, about 350 mg/mL, about 360 mg/mL, about 370 mg/mL, about 380 mg/mL, about 390 mg/mL, about 400 mg/mL, about 410 mg/mL, about 420 mg/mL, about 430 mg/mL, about 440 mg/mL, about 450 mg/mL, about 460 mg/mL, about 470 mg/mL, about 480 mg/mL, about 490 mg/mL, and about 500 mg/mL, including all ranges and values therebetween.

In some embodiments, the compounds of Formula I, Formula IA, Formula II, and Formula IIA, when administered to a subject in need thereof, provide a mean area under the curve (AUC₀₋₂₄) plasma concentration profile within the range of 1 h*ng/mL to about 50000 h*mg/mL, e.g., about 1 h*ng/mL, about 10 h*ng/mL, 20 h*ng/mL, about 30 h*ng/mL, about 40 h*ng/mL, about 50 h*ng/mL, about 60 h*ng/mL, about 70 h*ng/mL, about 80 h*ng/mL, about 90 h*ng/mL, about 100 h*ng/mL, about 110 h*ng/mL, about 120 h*ng/mL, about 130 h*ng/mL, about 140 h*ng/mL, about 150 h*ng/mL, about 200 h*ng/mL, about 300 h*ng/mL, about 400 h*ng/mL, about 500 h*ng/mL, about 600 h*ng/mL, about 700 h*ng/mL, about 800 h*ng/mL, about 900 h*ng/mL, about 1000 h*ng/mL, about 1100 h*ng/mL, about 1200 h*ng/mL, about 1300 h*ng/mL, about 1400 h*ng/mL, about 1500 h*ng/mL, about 1600 h*ng/mL, about 1700 h*ng/mL, about 1800 h*ng/mL, about 1900 h*ng/mL, about 2000 h*ng/mL, about 2100 h*ng/mL, about 2200 h*ng/mL, about 2300 h*ng/mL, about 2400 h*ng/mL, about 2500 h*ng/mL, about 2600 h*ng/mL, about 2700 h*ng/mL, about 2800 h*ng/mL, about 2900 h*ng/mL, about 3000 h*ng/mL, about 3100 h*ng/mL, about 3200 h*ng/mL, about 3300 h*ng/mL, about 3400 h*ng/mL, about 3500 h*ng/mL, about 3600 h*ng/mL, about 3700 h*ng/mL, about 3800 h*ng/mL, about 3900 h*ng/mL, about 4000 h*ng/mL, about 5000 h*ng/mL, about 6000 h*ng/mL, about 7000 h*ng/mL, about 8000 h*ng/mL, about 9000 h*ng/mL, about 10000 h*ng/mL, about 20000 h*ng/mL, about 30000 h*ng/mL, about 40000 h*ng/mL, about 50000 h*ng/mL, about 60000 h*ng/mL, about 70000 h*ng/mL, about 80000 h*ng/mL, about 90000 h*ng/mL, about 100000 h*ng/mL, about 100000 h*ng/mL, about 200000 h*ng/mL, about 300000 h*ng/mL, about 400000 h*ng/mL, about 500000 h*ng/mL, about 600000 h*ng/mL, about 700000 h*ng/mL, about 800000 h*ng/mL, about 900000 h*ng/mL, about 1 h*mg/mL, about 10 h*mg/mL, 20 h*mg/mL, about 30 h*mg/mL, about 40 h*mg/mL, about 50 h*mg/mL, about 60 h*mg/mL, about 70 h*mg/mL, about 80 h*mg/mL, about 90 h*mg/mL, about 100 h*mg/mL, about 110 h*mg/mL, about 120 h*mg/mL, about 130 h*mg/mL, about 140 h*mg/mL, about 150 h*mg/mL, about 160 h*mg/mL, about 170 h*mg/mL, about 180 h*mg/mL, about 190 h*mg/mL, about 200 h*mg/mL, about 210 h*mg/mL, about 220 h*mg/mL, about 230 h*mg/mL, about 240 h*mg/mL, about 250 h*mg/mL, about 260 h*mg/mL, about 270 h*mg/mL, about 280 h*mg/mL, about 290 h*mg/mL, about 300 h*mg/mL, about 310 mg/mL, about 320 h*mg/mL, about 330 h*mg/mL, about 340 h*mg/mL, about 350 h*mg/mL, about 360 h*mg/mL, about 370 h*mg/mL, about 380 h*mg/mL, about 390 h*mg/mL, about 400 h*mg/mL, about 410 h*mg/mL, about 420 h*mg/mL, about 430 h*mg/mL, about 440 h*mg/mL, about 450 h*mg/mL, about 460 h*mg/mL, about 470 h*mg/mL, about 480 h*mg/mL, about 490 h*mg/mL, and about 500 h*mg/mL, about 510 h*mg/mL, about 520 h*mg/mL, about 530 h*mg/mL, about 540 h*mg/mL, about 550 h*mg/mL, about 560 h*mg/mL, about 570 h*mg/mL, about 580 h*mg/mL, about 590 h*mg/mL, about 600 h*mg/mL, about 610 h*mg/mL, about 620 h*mg/mL, about 630 h*mg/mL, about 640 h*mg/mL, about 650 h*mg/mL, about 660 h*mg/mL, about 670 h*mg/mL, about 680 h*mg/mL, about 690 h*mg/mL, about 700 h*mg/mL, about 710 h*mg/mL, about 720 h*mg/mL, about 730 h*mg/mL, about 740 h*mg/mL, about 750 h*mg/mL, about 760 h*mg/mL, about 770 h*mg/mL, about 780 h*mg/mL, about 790 h*mg/mL, about 800 h*mg/mL, about 810 h*mg/mL, about 820 h*mg/mL, about 830 h*mg/mL, about 840 h*mg/mL, about 850 h*mg/mL, about 860 h*mg/mL, about 870 h*mg/mL, about 880 h*mg/mL, about 890 h*mg/mL, about 900 h*mg/mL, about 910 h*mg/mL, about 920 h*mg/mL, about 930 h*mg/mL, about 940 h*mg/mL, about 950 h*mg/mL, about 960 h*mg/mL, about 970 h*mg/mL, about 980 h*mg/mL, about 990 h*mg/mL, about 1000 h*mg/mL, about 1200 h*mg/mL, about 1300 h*mg/mL, about 1400 h*mg/mL, about 1500 h*mg/mL, about 1600 h*mg/mL, about 1700 h*mg/mL, about 1800 h*mg/mL, about 1900 h*mg/mL, about 2000 h*mg/mL, about 3000 h*mg/mL, about 4000 h*mg/mL, about 5000 h*mg/mL, about 6000 h*mg/mL, about 7000 h*mg/mL, about 8000 h*mg/mL, about 9000 h*mg/mL, about 10000 h*mg/mL, about 11000 h*mg/mL, about 12000 h*mg/mL, 13000 h*mg/mL, about 14000 h*mg/mL, about 15000 h*mg/mL, about 16000 h*mg/mL, about 17000 h*mg/mL, about 18000 h*mg/mL, about 19000 h*mg/mL, about 20000 h*mg/mL, about 21000 h*mg/mL, about 22000 h*mg/mL, 23000 h*mg/mL, about 24000 h*mg/mL, about 25000 h*mg/mL, about 26000 h*mg/mL, about 27000 h*mg/mL, about 28000 h*mg/mL, about 29000 h*mg/mL, about 30000 h*mg/mL, about 31000 h*mg/mL, about 32000 h*mg/mL, 33000 h*mg/mL, about 34000 h*mg/mL, about 35000 h*mg/mL, about 36000 h*mg/mL, about 37000 h*mg/mL, about 38000 h*mg/mL, about 39000 h*mg/mL, about 40000 h*mg/mL, about 41000 h*mg/mL, about 42000 h*mg/mL, 43000 h*mg/mL, about 44000 h*mg/mL, about 45000 h*mg/mL, about 46000 h*mg/mL, about 47000 h*mg/mL, about 48000 h*mg/mL, about 49000 h*mg/mL, about 50000 h*mg/mL, including all ranges and values therebetween.

In some embodiments, the method entails administering 2,2-dimethylbutyric acid, or a CoA ester or carnitine ester thereof, or a pharmaceutically acceptable salt, ester, or solvate thereof, at a concentration ranging from about, including about 2/mg/kg to 50 mg/kg. Blood plasma concentrations of 2,2-dimethylbutyric acid following administration were observed to be dose proportional. For example, the mean Cmax values were measured as 156 μg/mL, 203 μg/mL, and 256 μg/mL for 30, 40, and 50 mg/kg doses, respectively. In some embodiments, after administering 30 mg/kg of 2,2-dimethylbutyric acid, the patient's mean Cmax ranges from 80-125% of 156 μg/mL. In some embodiments, after administering 40 mg/kg of 2,2-dimethylbutyric acid, the patient's mean Cmax ranges from 80-125% of 203 μg/mL. In some embodiments, after administering 50 mg/kg of 2,2-dimethylbutyric acid, the patient's mean Cmax ranges from 80-125% of 256 μg/mL.

In some embodiments, after administering about 30-50 mg/kg of 2,2-dimethylbutyric acid to treat one or more of the metabolic diseases disclosed herein (e.g., MMA, IVA, or PA), the patient has a mean blood plasma concentration within about 80%-125% of the range of about 150-260 μg/mL, e.g., about 100 μg/mL, about 105 μg/mL, about 110 μg/mL, about 115 μg/mL, about 120 μg/mL, about 125 μg/mL, about 130 μg/mL, about 135 μg/mL, about 140 μg/mL, about 145 μg/mL, about 150 μg/mL, about 155 μg/mL, about 160 μg/mL, about 165 μg/mL, about 170 μg/mL, about 175 μg/mL, about 180 μg/mL, about 185 μg/mL, about 190 μg/mL, about 195 μg/mL, about 200 μg/mL, about 205 μg/mL, about 210 μg/mL, about 215 μg/mL, about 220 μg/mL, about 225 μg/mL, about 230 μg/mL, about 235 μg/mL, about 240 μg/mL, about 245 μg/mL, about 250 μg/mL, about 255 μg/mL, about 260 μg/mL, about 265, about 270 μg/mL, about 285 μg/mL, about 290 μg/mL, about 295 μg/mL, about 300 μg/mL, about 305 μg/mL, about 310 μg/mL, about 315 μg/mL, about 320 μg/mL, about 325 μg/mL, about 330 μg/mL, about 345 μg/mL, and about 350 μg/mL, including all ranges and values therebetween.

In some embodiments, after administering a therapeutically effective dose of 2,2-dimethylbutyric acid, the patient has a steady state blood plasma concentration within the range of from about 50-500 μg/mL, e.g., 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 165 μg/mL, about 170 μg/mL, about 175 μg/mL, about 180 μg/mL, about 185 μg/mL, about 190 μg/mL, about 195 μg/mL, about 200 μg/mL, about 200 μg/mL, about 205 μg/mL, about 210 μg/mL, about 215 μg/mL, about 220 μg/mL, about 225 μg/mL, about 230 μg/mL, about 235 μg/mL, about 240 μg/mL, about 245 μg/mL, about 250 μg/mL, about 255 μg/mL, about 260 μg/mL, about 265, about 270 μg/mL, about 285 μg/mL, about 290 μg/mL, about 295 μg/mL, about 300 μg/mL, about 305 μg/mL, about 310 μg/mL, about 315 μg/mL, about 320 μg/mL, about 325 μg/mL, about 330 μg/mL, about 335 μg/mL, about 340 μg/mL, about 345 μg/mL, about 350 μg/mL, about 355 μg/mL, about 360 μg/mL, about 365, about 370 μg/mL, about 385 μg/mL, about 390 μg/mL, about 395 μg/mL, about 400 μg/mL, about 405 μg/mL, about 410 μg/mL, about 415 μg/mL, about 420 μg/mL, about 425 μg/mL, about 430 μg/mL, about 435 μg/mL, about 440 μg/mL, about 445 μg/mL, about 450 μg/mL, about 455 μg/mL, about 460 μg/mL, about 465, about 470 μg/mL, about 485 μg/mL, about 490 μg/mL, about 495 μg/mL, and about 500 ng/mL, including all ranges and values therebetween.

Mean AUC values for 2,2-dimethylbutyric acid were observed to be 2182, 2625, and 3196 h*mg/mL for 30, 40, and 50 mg/kg doses, respectively. In some embodiments, after administering 30 mg/kg of 2,2-dimethylbutyric acid, the patient's mean AUC ranges from 80-125% of 2182 μg/mL. In some embodiments, after administering 40 mg/kg of 2,2-dimethylbutyric acid, the patient's mean AUC ranges from 80-125% of 2625 μg/mL. In some embodiments, after administering 50 mg/kg of 2,2-dimethylbutyric acid, the patient's mean AUC ranges from 80-125% of 3196 μg/mL. In some embodiments, after administering about 30-50 mg/kg of 2,2-dimethylbutanoic acid, the patient has a mean AUC within about 80%-125% of the range of about 2000-3200 μg/mL h*mg/mL, e.g., about 1500 h*μg/mL, about 1600 h*μg/mL, about 1700 h*μg/mL, about 1800 h*μg/mL, about 1900 h*μg/mL, about 2000 h*μg/mL, about 2100 h*μg/mL, about 2200 h*μg/mL, about 2300 h*μg/mL, about 2400 h*μg/mL, about 2500 h*μg/mL, about 2600 h*μg/mL, about 2700 h*μg/mL, about 2800 h*μg/mL, about 2900 h*μg/mL, about 3000 h*μg/mL, about 3100 h*μg/mL, about 3200 h*μg/mL, about 3300 h*μg/mL, about 3400 h*μg/mL, about 3500 h*μg/mL, about 3600 h*μg/mL, about 3700 h*μg/mL, about 3800 h*μg/mL, about 3900 h*μg/mL, and about 4000 h*μg/mL, about 4100 h*μg/mL, about 4200 h*μg/mL, about 4300 h*μg/mL, about 4400 h*μg/mL, and about 4500 h*μg/mL, inclusive of all values and subranges therebetween.

### Pharmaceutical Compositions

In further embodiments of the present disclosure, pharmaceutical compositions comprising one or more compounds disclosed herein, or a pharmaceutically acceptable solvate, ester, metabolite, or salt thereof, and a pharmaceutically acceptable excipient or adjuvant is provided. The pharmaceutically acceptable excipients and adjuvants are added to the composition or formulation for a variety of purposes. In other embodiments, pharmaceutical compositions comprising one or more compounds disclosed herein, or a pharmaceutically acceptable solvate, ester, metabolite, or salt thereof, and further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

For the purposes of this disclosure, the compounds of the present disclosure can be formulated for administration by a variety of means including orally and parenterally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The compounds disclosed herein can be formulated in accordance with the routine procedures adapted for desired administration route. Accordingly, the compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

In certain embodiments, a pharmaceutical composition of the present disclosure is prepared using known techniques, including, but not limited to mixing, dissolving, granulating, dragee-making, levigating, emulsifying, encapsulating, entrapping or tableting processes.

In some embodiments, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M phosphate buffer or saline (e.g., about 0.8%). Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

In various embodiments, the pharmaceutical composition may be selected from the group consisting of a solid, powder, liquid and a gel. In certain embodiments, the pharmaceutical compositions of the present disclosure is a solid (e.g., a powder, tablet, a capsule, granulates, and/or aggregates). In certain of such embodiments, the solid pharmaceutical composition comprises one or more excipients known in the art, including, but not limited to, starches, sugars, diluents, granulating agents, lubricants, binders, and disintegrating agents.

Solid pharmaceutical compositions that are compacted into a dosage form, such as a tablet, may include excipients whose functions include helping to bind the active ingredient and other excipients together after compression. Binders for solid pharmaceutical compositions and/or combinations include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition and/or combination. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and/or combination and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition and/or combination to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that may be included in the composition and/or combination of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In certain embodiments, a pharmaceutical composition of the present invention is a liquid (e.g., a suspension, elixir and/or solution). In certain of such embodiments, a liquid pharmaceutical composition is prepared using ingredients known in the art, including, but not limited to, water, glycols, oils, alcohols, flavoring agents, preservatives, and coloring agents.

Liquid pharmaceutical compositions can be prepared using compounds of the present disclosure, and any other solid excipients where the components are dissolved or suspended in a liquid carrier such as water, vegetable oil, alcohol, polyethylene glycol, propylene glycol, or glycerin.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Liquid pharmaceutical compositions can contain emulsifying agents to disperse uniformly throughout the composition and/or combination an active ingredient or other excipient that is not soluble in the liquid carrier. Emulsifying agents that may be useful in liquid compositions and/or combinations of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition can also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

In one embodiment, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables. Formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

In certain embodiments, a pharmaceutical compositions of the present invention are formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition of the present invention comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

Appropriate pharmaceutical compositions of the present disclosure can be determined according to any clinically-acceptable route of administration of the composition to the subject. The manner in which the composition is administered is dependent, in part, upon the cause and/or location. One skilled in the art will recognize the advantages of certain routes of administration. The method includes administering an effective amount of one or more compounds of the present disclosure (or composition comprising such) to achieve a desired biological response, e.g., an amount effective to alleviate, ameliorate, or prevent, in whole or in part, a symptom of a condition to be treated, e.g., metabolic disorders. In various embodiments, the route of administration is systemic, e.g., oral or by injection.

In certain embodiments, the pharmaceutical compositions of the present disclosure are prepared for oral administration. In certain of such embodiments, the pharmaceutical compositions are formulated by combining one or more agents and pharmaceutically acceptable carriers. Certain of such carriers enable pharmaceutical compositions to be formulated as tablets, pills, dragees, capsules, liquids, gels, syrups, slurries, suspensions and the like, for oral ingestion by a subject. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical compositions are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical compositions for oral administration are push-fit capsules made of gelatin. Certain of such push-fit capsules comprise one or more pharmaceutical agents of the present invention in admixture with one or more filler such as lactose, binders such as starches, and/or lubricants such as talc or magnesium stearate and, optionally, stabilizers. In certain embodiments, the pharmaceutical compositions for oral administration are soft, sealed capsules made of gelatin and a plasticizer, such as glycerol or sorbitol. In certain soft capsules, one or more compounds disclosed herein are be dissolved or suspended in suitable liquids, such as fatty oils, liquid paraffin, or liquid polyethylene glycols. In addition, stabilizers may be added.

In other embodiments the compound of the present disclosure are administered by the intravenous route. In further embodiments, the parenteral administration may be provided in a bolus or by infusion.

In various aspects, the amount of the compound disclosed herein, or a pharmaceutically acceptable solvate, ester, metabolite, or salt thereof, can be administered at about 0.001 mg/kg to about 100 mg/kg body weight (e.g., about 0.01 mg/kg to about 10 mg/kg or about 0.1 mg/kg to about 5 mg/kg).

In some embodiments, the compounds of the disclosure are formulated in a composition disclosed in U.S. Pat. No. 8,242,172, in order to improve the physiological stability of the compound. Physiological stable compounds are compounds that do not break down or otherwise become ineffective upon introduction to a patient prior to having a desired effect. Compounds are structurally resistant to catabolism, and thus, physiologically stable, or coupled by electrostatic or covalent bonds to specific reagents to increase physiological stability. Such reagents include amino acids such as arginine, glycine, alanine, asparagine, glutamine, histidine or lysine, nucleic acids including nucleosides or nucleotides, or substituents such as carbohydrates, saccharides and polysaccharides, lipids, fatty acids, proteins, or protein fragments. Useful coupling partners include, for example, glycol such as polyethylene glycol, glucose, glycerol, glycerin and other related substances.

Physiological stability can be measured from a number of parameters such as the half-life of the compound or the half-life of active metabolic products derived from the compound. Certain compounds of the invention have in vivo half-lives of greater than about fifteen minutes, preferably greater than about one hour, more preferably greater than about two hours, and even more preferably greater than about four hours, eight hours, twelve hours or longer. Although a compound is stable using this criteria, physiological stability cam also be measured by observing the duration of biological effects on the patient. Clinical symptoms which are important from the patient's perspective include a reduced frequency or duration, or elimination of the need for oxygen, inhaled medicines, or pulmonary therapy.

The concentration of a disclosed compound in a pharmaceutically acceptable mixture will vary depending on several factors, including the dosage of the compound to be administered, the pharmacokinetic characteristics of the compound(s) employed, and the route of administration. The agent may be administered in a single dose or in repeat doses. The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. Treatments may be once administered daily or more frequently depending upon a number of factors, including the overall health of a patient, and the formulation and route of administration of the selected compound(s).

The compounds or pharmaceutical compositions of the present disclosure may be manufactured and/or administered in single or multiple unit dose forms.

### Methods of Treatment

As discussed herein, the compounds of Formula I, IA, II, and/or HA can be administered to a patient to treat an organic acidemia disclosed herein.

In some embodiments, the compounds of Formula I, IA, II, and/or IIA administered to a patient in need thereof according to the methods disclosed herein are provided single or divided (e.g., three times in a 24 hour period) doses, wherein the amount for each of the three doses is determined by patient weight. According to a weight-based dosing regimen, each dose administered may be in a range of from about 0.1 mg/kg to about 500 mg/kg, e.g., about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 12 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 100 mg/kg, about 150 mg/kg, about 200 mg/kg, about 250 mg/kg, about 300 mg/kg, about 350 mg/kg, about 400 mg/kg, about 450 mg/kg, and about 500 mg/kg, inclusive of all values and subranges therebetween. In some embodiments, the dose is in a range of from about 0.1 mg/kg to about 10 mg/kg. In some embodiments, the dose is less than about 10 mg/kg. In some embodiments, the dose is in the range of from about 1 mg to about 100 g, e.g., about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1 g, about 2 g, about 3 g, about 4 g, about 5 g, about 6 g, about 7 g, about 8 g, about 9 g, about 10 g, about 15 g, about 20 g, about 25 g, about 30 g, about 35 g, about 40 g, about 45 mg, about 50 g, about 55 g, about 60 g, about 65 g, about 70 g, about 75 g, about 80 g, about 85 g, about 90 g, about 95 g, and about 100 g, inclusive of all values and subranges therebetween. Any of the above doses may a “therapeutically effective” amount as used herein.

In some embodiments, one or more compounds disclosed herein may be administered one or more times a day, e.g., 1, 2, 3, 4, 5, 6, 7, 8, 9, or 10 times a day. In some embodiments, one or more compounds disclosed herein may administered to the patient for a period of time sufficient to efficacious for the treatment of an organic acidemia. In some embodiments, the treatment regimen is an acute regimen. In some embodiments, the treatment regimen is a chronic treatment regimen. In some embodiments, the patient is treated for 1 week, about 2 weeks, about 3 weeks, about 4 weeks, about 5 weeks, about 6 weeks, about 7 weeks, about 8 weeks, about 9, weeks about 10 weeks, about 20 weeks, about 30 weeks, about 40 weeks, about 50 weeks, about 60 weeks, about 70 weeks, about 80 weeks, about 90 weeks, about 100 weeks, about 1 year, about 2 years, about 3 years, about 4 years, about 5 years, about 6 years, about 7 years, about 8 years, about 9 years, about 10 years, about 15 years, about 20 years, about 30 years, about 40 years, about 50 years, about 60 years, about 70 years, about 80 years, or for the entirety of the patient's lifetime.

In some embodiments, the patient treated accordance with the methods provided herein is a newborn, or is about 1 month to 12 months old, about 1 year to 10 years old, about 10 to 20 years old, about 12 to 18 years old, about 20 to 30 years old, about 30 to 40 years old, about 40 to 50 years old, about 50 to 60 years old, about 60 to 70 years old, about 70 to 80 years old, about 80 to 90 years old, about 90 to 100 years old, or any age in between. In some embodiments, a patient treat in accordance with the methods disclosed herein is a newborn human. In some embodiments, the patient treated in accordance with the methods provided herein is between the age of newborn and 1 year old. In some embodiments, patient is between the age of 1 year old and 18 years old. In some embodiments, the patient is between the age of 1 year old and 5 years old. In some embodiments, the patient is between the age of 5 years old or 12 years old. In some embodiments, the patient is between the age of 12 years old and 18 years old. In some embodiments, the patient is at least 1 year old or older. In some embodiments, the patient is at least 2 years old or older. In some embodiments, the patient is between the ages of 2 years old and 5 years old, 2 years old and 10 years old, 2 years old and 12 years old, 2 years old and 15 years old, 2 years old and 18 years old, 5 years old and 10 years old, 5 years old and 12 years old, 5 years old and 15 years old or 5 years old and 18 years old.

In some embodiments, the patient is a pediatric (12 and under), an adolescent (13 to 17), an adult (18 to 65), or a geriatric (65 or older). In some embodiments, the pediatric patient is a new born child, e.g., from 0 to 6 months. In some embodiments, the pediatric patient is an infant, aged 6 months to 1 year. In some embodiments, the pediatric patient is 6 months to 2 years old. In some embodiments, the pediatric patient is 2 years to 6 years old. In some embodiments, the pediatric patient is 6 years to 12 years old. In some embodiments, the child is under 10 years of age.

In some embodiments, the methods for treating the diseases provided herein improve or developmental or cognitive function in a subject. Such improvements in developmental or cognitive function may be as assessed by, e.g., the Bayley Scale of Infant Development, Wechsler Preschool and Primary Scale of Intelligence (WIPPSI), Wechsler Intelligence Scale for Children (WISC) or Wechsler Adult Intelligence Scale (WAIS). Some embodiments, an improvement in developmental or cognitive function may be assessed using the methods provided in the examples in US 2014/0343009, which is herein incorporated by reference in its entirety for all purposes.

In some embodiments, the methods provided herein improve control of muscle contractions by a patient as assessed by methods well known in the art, e.g., the Burke-Fahn-Marsden rating scale. In certain aspects, the methods provided herein decrease the occurrence of metabolic decompensation episodes, characterized by, e.g., vomiting, hypotonia, and alteration in consciousness.

In some embodiments, the methods provided herein are suitable in patients that have received a liver transplant (e.g., OLT) or a kidney transplant or a liver and kidney transplant.

In some embodiments, the methods provided herein improve renal function. In certain embodiments, the methods provided herein decrease the need for kidney transplant, liver transplant or both.

In some aspects, the methods provided herein decrease the requirement for hospitalization. In certain embodiments, the methods provided herein decrease the length and/or frequency of hospitalization.

In some embodiments, such methods reduce the production of metabolites in a subject. Advantageously and surprisingly, the compounds and methods of the present disclosure are able to reduce the production of toxic metabolites in various tissue throughout the body in order to achieve disease remediation. In some embodiments, the metabolites are metabolites produced in the liver. In some embodiments, the metabolites are metabolites produced in the muscle. In some embodiments, the metabolites are metabolites produced in the brain. In some embodiments, the metabolites are metabolites produced in the kidney. In some embodiments, the metabolites are metabolites produced in any organ tissue. In some embodiments, the metabolite is a metabolite of one or more of a branched chain amino acid, methionine, threonine, odd-chain fatty acids and cholesterol. In some embodiments, the metabolites can be propionyl-CoA. In some embodiments, the metabolite is methylmalonyl-CoA. In some embodiments, the metabolite is 2-methylcitric acid (MCA).

In some embodiments, upon the administration of one or more compounds of Formula I, IA, II, or IIA (or a derivative, metabolite, or pharmaceutically acceptable salt thereof), at least one metabolite of a branched chain amino acid is (e.g., propionyl-CoA and/or methylmalonyl-CoA levels) is reduced by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, or by any values in between. In some embodiments, the level can be reduced by at least 87.5%. In some embodiments, at least one metabolite of a branched chain amino acid (e.g., propionyl-CoA and/or methylmalonyl-CoA levels) is reduced by an amount ranging from about 1% to about 100%, e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, inclusive of all values and subranges therebetween. In some embodiments, the metabolite is a metabolite of one or more of a branched chain amino acid, methionine, threonine, odd-chain fatty acids and cholesterol. In some embodiments, the metabolite (or metabolites), such as propionyl-CoA and/or methylmalonyl-CoA, are reduced to a level that achieves the therapeutic effects in treating organic acidemia. In some embodiments, the metabolite is propionyl-CoA and/or methylmalonyl-CoA. In some embodiments, the metabolite is 3-hydroxypropionic acid, methylcitrate, methylmalonic acid, propionylglycine, or propionylcarnitine, or combinations thereof. In some embodiments, metabolite is 2-ketoisocaproate, isovaleryl-CoA, 3-methylcrotonyl-CoA, 3-methylglutaconyl-CoA, 3-OH-3-methylglutaryl-CoA, 2-keto-3-methylvalerate, 2-methylbutyryl-CoA, tiglyl-CoA, 2-methyl-3-OH-butyryl-CoA, 2-methyl-acetoacetyl-CoA, 2-ketoisovalerate, isobutyryl-CoA, methylacrylyl-CoA, 3-OH-isobutyryl-CoA, 3-OH-isobutyrate, methylmalonic semialdehyde, propionyl-CoA, or methylmalonyl-CoA, or combinations thereof

In one embodiment, the compounds of the present disclosure (or pharmaceutically acceptable salt, ester, metabolite, or solvate thereof) is administered to the subject in a composition. The “composition” as described herein refers to a mixture that contains at least one pharmaceutically acceptable compound and at least one pharmaceutically acceptable carrier. In one embodiment, the composition contains an effective amount of at least one pharmaceutically acceptable compound. In embodiments, an effective amount of an inhibitor is administered to the subject.

The compounds of the disclosure may be administered by any appropriate route of administration. As previously defined, the route includes, but is not limited to oral, parenteral, intramuscular, transdermal, intravenous, inter-arterial, nasal, vaginal, sublingual, and subungual. Further, the route also includes, but is not limited to auricular, buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrachronchial, intrabursal, intracardiac, intracartilagenous, intracaudal, intracavernous, intracavitary, intracerebral, intracisternal, intracorneal, intracoronary, intracorporus cavernosum, intradermal, intradiscal, intraducatal, intraduodenal, intradural, intraepidermal, intraesophageal, intragastric, intragingival, intraileal, intralesional, intralumical, intralymphatic, intramedullary, intrameningeal, intraocular, intraovarian, intrapericardial, intraperitoneal, intrapleural, intrapulmonary, intrasinal, intrasynovial, intratendinous, intratesticular, intrathecal, intrathoracic, intratubular, intratumor, intratympanic, intrauterine, intravascular, intravenous bolus, intravenous drip, intraventricular, intravesical, intravitreal, iontophoresis, irrigation, laryngeal, nasal, nasogastric, occlusive dressing technique, ophthalmic, oropharyngeal, percutaneous, periarticular, peridural, periodontal, rectal, respiratory, retrobulbar, soft tissue, subarachnoid, subconjunctival, subcutaneous, submucosal, topical, transmucosal, transplacental, transtracheal, transtympanic, ureteral, or urethal.

The methods of the present disclosure can be combined with other therapies used in the treatment of metabolic diseases (including organic acidemias, e.g., PA or MMA) which can be administering subsequently, simultaneously, or sequentially (e.g., before or after) with the compounds of Formula I, IA, II, or IIA (e.g., 2,2-dimethylbutyric acid, or CoA esters or carnitine esters thereof, or pharmaceutically acceptable salts, solvates, or esters thereof). Non-limiting examples of additional therapeutic agent which can be combined with the methods disclosed herein include: L-carnitine; glucose; L-arginine; Polycal (maltodextrin-based carbohydrate supplement); ammonia scavengers used to treat acute hyperammonemia, such as N-carbamyl-glutamate, sodium benzoate, sodium phenyl acetate, sodium phenylbutyrate, glycerol phenylbutyrate; antibiotics used to reduce the intestinal flora, such as metronidazole, amoxicillin or cotrimoxazole; vitamin B₁₂ (in B₁₂-responsive MMA patients); biotin; growth hormone therapy; low-protein diets; antioxidant therapies, such as N-acetylcysteine, cysteamine or α-tocotrienol quinone; and anaplerotic therapies, such as citrate, glutamine, ornithine α-ketoglutarate or prodrugs of succinate; and essential amino acids such as norvaline, methionine, isoleucine, or threonine. In some embodiments, the additional therapeutic agent which can be combined with the methods disclosed herein is a messenger RNA therapeutic. In some embodiments, the messenger RNA therapeutic is mRNA-3927 or mRNA-3704. mRNA-3927 includes two mRNAs that encode for the alpha and beta subunits of the mitochondrial enzyme propionyl-CoA carboxylase (PCC), encapsulated within a lipid nanoparticle (LNP) and can be used to restore missing or dysfunctional proteins that cause PA. mRNA-3704 consists of mRNA encoding human MUT, the mitochondrial enzyme commonly deficient in MMA, encapsulated within a LNP. It is contemplated that the compounds of the present disclosure can be combined with mRNA-3927 or mRNA-3704 therapy, because the compounds of the present disclosure will reduce the levels of toxic metabolites disclosed herein, whereas mRNA-3927 or mRNA-3704 is target primarily the liver. In some embodiments, the compounds of the present disclosure may be used in patient with an organic acidemia after said patients receives a liver transplant. In some embodiments, the compounds of the disclosure are administered in combination with an AAV therapy, such as the AAV therapy from LogicBio (LB-001).

### EXAMPLES

The following examples are offered by way of illustration and not by way of limitation.

### List of Abbreviations and Definitions of Terms

| Abbreviation | Definition |
|---|---|
| ACDH | acyl-CoA dehydrogenase |
| AUC | area under the concentration time curve |
| C2 | acetyl-carnitine |
| C3 | propionyl-carnitine |
| CL | clearance rate |
| C4 | methylmalonyl-carnitine |
| Cₘₐₓ | maximum concentration |
| CNS | central nervous system |
| CoASH | coenzyme A |
| CPS1 | carbamoyl phosphate synthetase I |
| CS | citrate synthase |
| DMBA | 2,2-dimethylbutanoic acid |
| GLP | Good Laboratory Practice |
| EC₅₀ | effective concentration, 50% |
| EC₉₀ | effective concentration, 90% |
| Compound 5-CoA | 2,2-dimethylbutyryl-coenzyme A |
| HT-MS/MS | high-throughput mass spectrometry |
| IRB | Institutional Review Board |
| MRM | multi-reactant monitoring |
| MSMS | tandem mass spectronmy |
| LC-MS/MS | liquid chromatography mass spectrometry |
| M-acid | methylmalonic acid |
| MCA | 2-methylcitric acid |
| M-CoA | methylmalonyl-CoA |
| MMA | methylmalonic acidemia |
| MUT | methylmalonyl-CoA mutase |
| NAGS | N-acetylglutamate synthase |
| OMIM | Online Mendelian Inheritance in Man |
| PA | propionic acidemia |
| PCC | propionyl-CoA carboxylase |
| PCCA | α subunit of propionyl-CoA carboxylase |
| PCCB | β subunit of propionyl-CoA carboxylase |
| P-CoA | propionyl coenzyme A |
| PDH | pyruvate dehydrogenase |
| pHeps | primary hepatocytes |
| PK | pharmacokinetics |
| SD | standard deviation |
| SIL | Stable isotope labeling |
| TCA | tricarboxylic acid |

### Example 1

Reduction of Propionyl-CoA Levels and Accumulation of CoA Ester with Compounds 1-7.

Primary hepatocytes are isolated from explanted livers of propionic acidemia patients and are cultured on day 1 using standard protocols (see: Chapman et al. “Recapitulation of metabolic defects in a model of propionic acidemia using patient-derived primary hepatocytes” Mol. Genet. Metab. 2016, 117(3), 355-362). Approximately 2×10⁵ hepatocytes are plated into each well of a collagen-coated 48-well tissue culture plate and pre-conditioned in a custom Modified Corning culture media for Hepatocells (Corning) without low levels of branched chain amino acids for 72 hours. On day 4, hepatocytes are treated with increasing doses of compound (0, 1, 3, 10, 30, 100, 300, 1000 μM) for 30 min. After 30 min the cells are challenged with ¹³C-isoleucine (3 mM). At the end of the challenge period, cells are lysed in 100 μL of 70%/6 acetonitrile (MeCN) and 0.1% trifluoroacetic acid (TFA) containing 100 μM of ethymalonyl-CoA as an internal standard. Cells are removed from the well by scraping into the lysis buffer. The collected cellular samples are then dispensed into a microfuge tube. The plate wells are washed again with lysis buffer to ensure the remaining cells are dislocated from the well. All remaining collected cells are then moved into the centrifuge tube. The cellular samples are flash frozen in liquid nitrogen and stored at −80° C. To begin processing, the frozen cellular lysates are thawed on ice and vortexed. The samples are centrifuged at 20,000 g for 10 min at 4° C. and the total volume of supernatant is transferred to a no-bind 96-well plate on ice. The samples are dried under vacuum for about 2 hours and then resuspended in 150 μL of water in each well. Total volumes of samples are filtered through a prepared Durapore filter plate into a no-bind 96-well plate. The filtered samples are stored at −80° C. for HTMS/MS analysis.

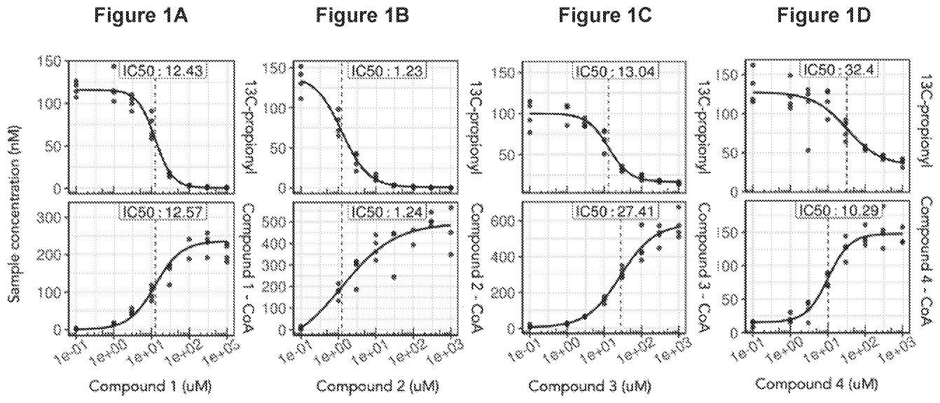

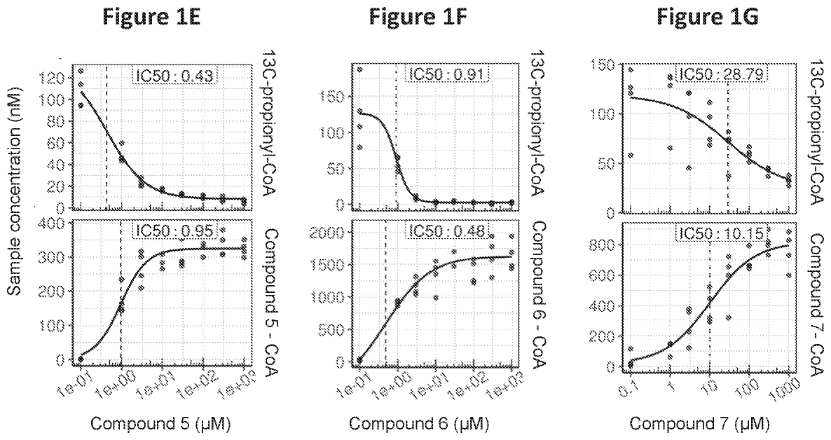

Treatment of primary hepatocytes isolated from livers of propionic acidemia patients with compounds 1-7 resulted in a dose-dependent reduction of intracellular propionyl-CoA (FIG. 1A-1F). Compounds reduced ¹³C-propioyl-CoA by >90% over the 1 hour treatment time. The EC₅₀ for the reduction in ¹³C-propionyl-CoA is similar to the EC₅₀ for the accumulation of the compound CoA ester.

### Example 2

Reduction of Propionyl-CoA Levels from all Sources Following Compound 1 Treatment in PA Primary Hepatocytes

Primary hepatocytes isolated from livers of propionic acidemia patients are treated with increasing doses of compound 1 (0, 1, 3, 10, 30, 100, 300, 1000 μM) for 30 min. After 30 min the cells are challenged with the different sources of P-CoA, which may include ¹³C-ketoisovaleric acid (KIVA) (1 mM), ¹⁷C-isoleucine (ILE) (3 mM), ¹³C-threonine (THR) (5 mM), ¹³C-methionine (MET) (5 mM), ¹³C-heptanoate (100 μM), or ¹³C-propionate (5 mM) for 60 minutes. At the end of the challenge period, media is removed and the cells are lysed with 70% MeCN and 0.1% TFA containing 100 μM of ethymalonyl-CoA as an internal standard and harvested. Cell lysates are processed for HTMS/MS analysis.

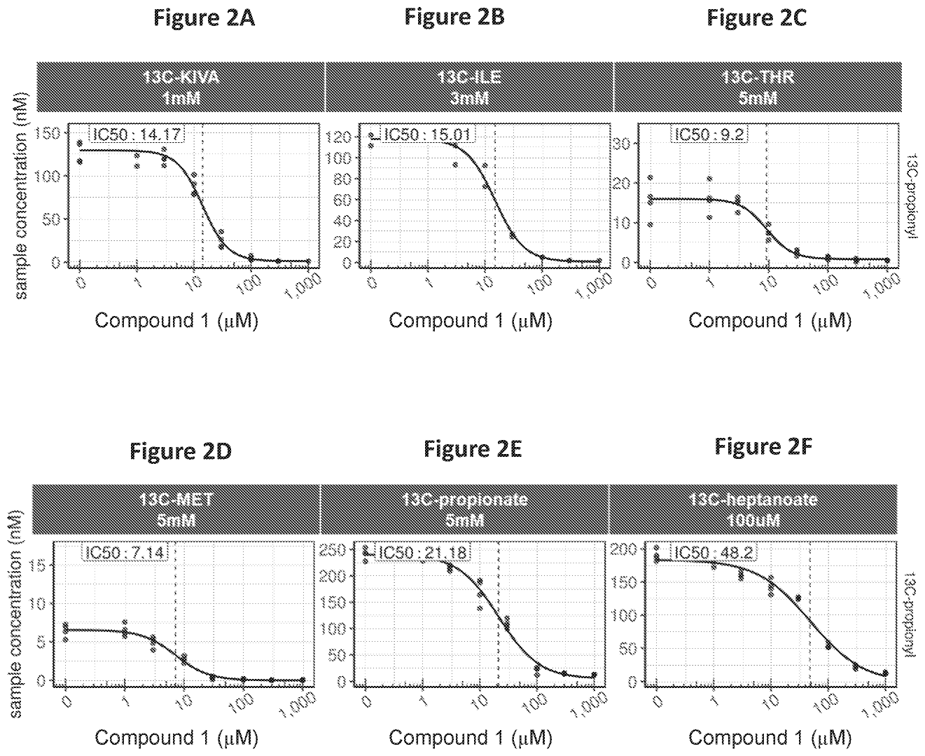

Treatment of primary hepatocytes isolated from livers of propionic acidemia patients with compound 1 resulted in a dose-dependent reduction of intracellular propionyl-CoA from all sources investigated (FIG. 2A-2F). This indicates that treatment with compound 1 alleviates the primarily metabolic defect (accumulation of propionyl-CoA) in primary hepatocytes of propionic acidemia patients.

### Example 3

Reduction of Propionyl-CoA Levels from all Sources Following Compound 5 Treatment in PA Primary Hepatocytes

Primary hepatocytes isolated from livers of propionic acidemia patients are treated with increasing doses of compound 5 (0, 0.1, 0.3, 1, 3, 10, 30, 100 μM) for 30 minutes. After 30 minutes the cells are challenged with the different sources of P-CoA, which may include ¹³C-KIVA (1 mM), ¹³C-isoleucine (3 mM), ¹³C-threonine (5 mM), ¹³C-methionine (5 mM), ¹³C-heptanoate (100 μM), or ¹³C-propionate (5 mM) for 60 minutes. At the end of the challenge period, media is removed and the cells are lysed with 70% MeCN and 0.1% TFA containing 100 μM of ethymalonyl-CoA as an internal standard and harvested. Cell lysates are processed for HTMS/MS analysis.

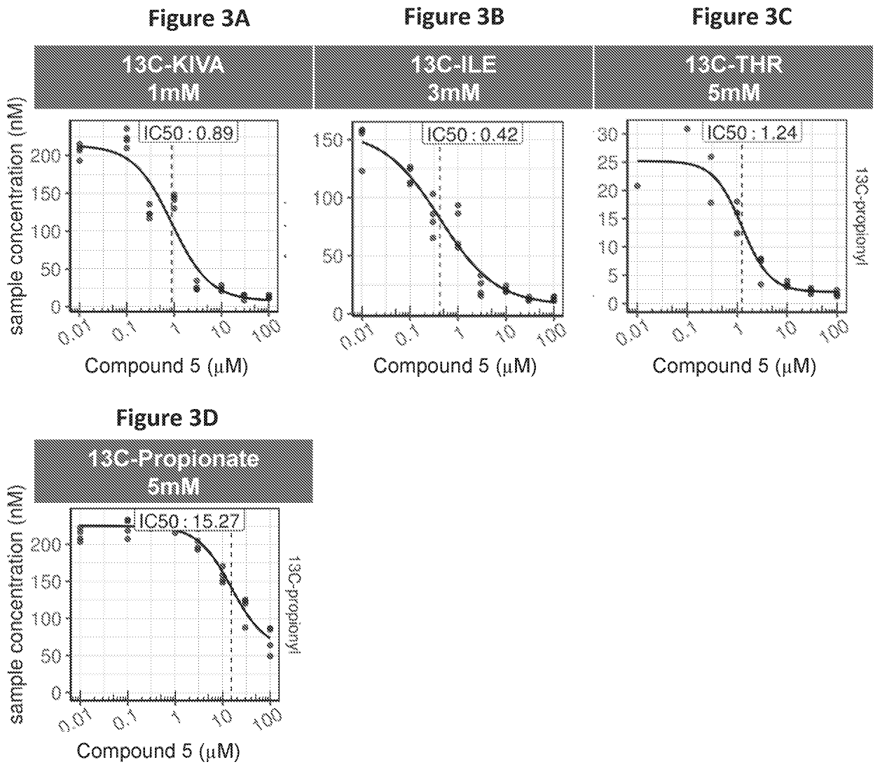

Treatment of primary hepatocytes isolated from livers of propionic acidemia patients with compound 5 resulted in a dose-dependent reduction of intracellular propionyl-CoA from all sources investigated (FIG. 3A-3D). This indicates that treatment with compound 5 alleviates the primarily metabolic defect (accumulation of propionyl-CoA) in primary hepatocytes of propionic acidemia patients.

### Example 4

Reduction of Methylmalonyl-CoA and Propionyl-CoA Levels from all Sources Following Compound 1 Treatment of MMA Primary Hepatocytes

Primary hepatocytes isolated from livers of methylmalonic acidemia patients are treated with increasing doses of compound 1 (0, 1, 3, 10, 30, 100, 300, 1000 μM) for 30 minutes. After 30 minutes the cells are challenged with the different sources of P-CoA, which may include ¹³C-KIVA (1 mM), ¹³C-isoleucine (3 mM), ¹³C-threonine (5 mM), ¹³C-methionine (5 mM), ¹³C-heptanoate (100 μM), or ¹³C-propionate (5 mM) for 60 minutes. At the end of the challenge period, media is removed and the cells are lysed with 70% MCCN and 0.1% TFA containing 100 μM of ethymalonyl-CoA as an internal standard and harvested. Cell lysates are processed for HTMS/MS analysis.

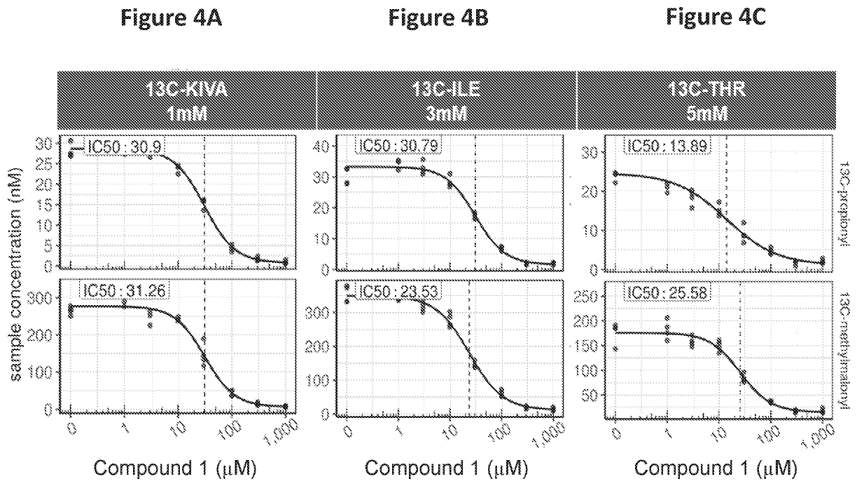

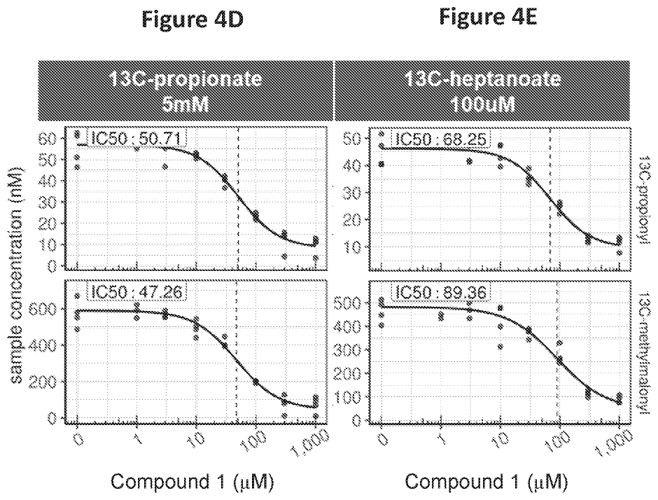

Treatment of primary hepatocytes isolated from livers of methylmalonic academia patients with compound 1 resulted in a dose-dependent reduction of intracellular propionyl-CoA and methylmalonyl-CoA from all sources investigated (FIG. 4A-4E). This indicates that treatment with compound 1 alleviates the primarily metabolic defect (accumulation of propionyl-CoA and methylmalonyl-CoA) in primary hepatocytes of methylmalonic acidemia patients.

### Example 5

Reduction of Methylmalonyl-CoA and Propionyl-CoA Levels from all Sources Following Compound 5 Treatment of MMA Primary Hepatocytes

Primary hepatocytes isolated from livers of methylmalonic acidemia patients are treated with increasing doses of compound 5 (0, 0.1, 0.3, 1, 3, 10, 30, 100 μM) for 30 min. After 30 minutes the cells are challenged with the different sources of P-CoA, which may include ¹³C-KIVA (1 mM), ¹³C-isoleucine (3 mM), ¹³C-threonine (5 mM), ¹³C-methionine (5 mM), ¹³C-heptanoate (100 μM), or ¹³C-propionate (5 mM) for 60 minutes. At the end of the challenge period, media is removed and the cells are lysed with 70% MeCN and 0.1% TFA containing 100 μM of ethymalonyl-CoA as an internal standard and harvested. Cell lysates are processed for HTMS/MS analysis.

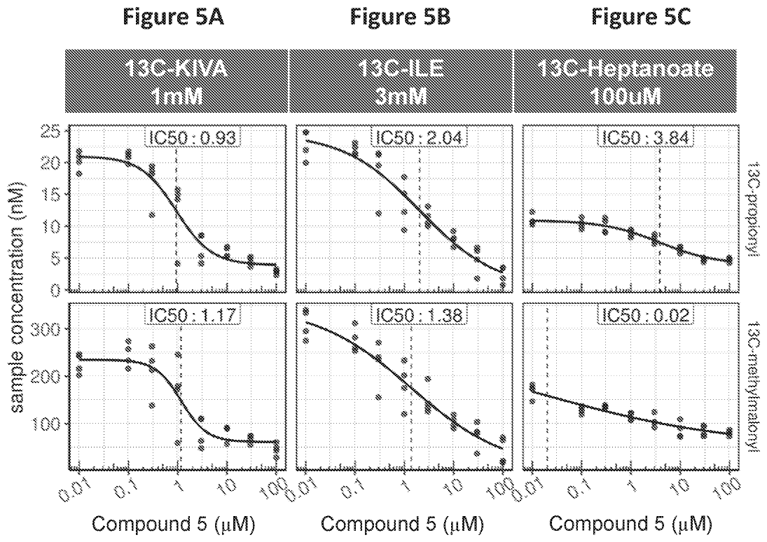

Treatment of primary hepatocytes isolated from livers of methylmalonic academia patients with compound 5 resulted in a dose-dependent reduction of intracellular propionyl-CoA and methylmalonyl-CoA from all sources investigated (FIG. 5A-5C). This indicates that treatment with compound 5 alleviates the primarily metabolic defect (accumulation of propionyl-CoA and methylmalonyl-CoA) in primary hepatocytes of methylmalonic acidemia patients.

### Example 6

### Reduction of Clinical Biomarker Propionyl-Carnitine (C3) Levels Following Compound 1 Treatment

Primary hepatocytes isolated from livers of propionic acidemia patients are treated with increasing doses of compound 1 (0, 1, 3, 10, 30, 100, 300, 1000 μM) for 30 min. After 30 min the cells are challenged with ¹³C-KIVA (1 mM) and ¹³C-isoleucine (3 mM) for 60 minutes. At the end of the challenge period, media is removed and the cells are washed with ice cold PBS. Cells are lysed with 70% MeCN containing 4 nM of hexanoyl-carnitine as an internal standard (lysis buffer), harvested and processed for HTMS/MS analysis.

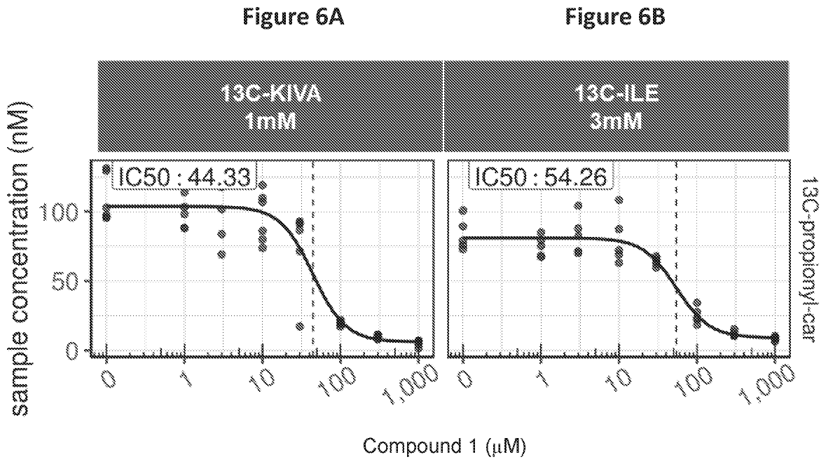

Treatment of primary hepatocytes isolated from livers of propionic acidemia patients with compound 1 resulted in a dose-dependent reduction of intracellular propionyl-carnitine (C3) from ¹³C-KIVA or ¹³C-isoleucine (FIG. 6A-6B). The resulting reduction in C3 in PA donor hepatocytes indicates that treatment with compound 1 has an impact on the primary diagnostic clinical biomarker in primary hepatocytes of propionic acidemia patients.

### Example 7

### Effect on Ureagenesis Following Compound 5 Treatment

For this experiment, we deployed the HemoShear REVEAL-Tx™ technology, which is based on the cone-and-plate configuration or viscometer combined with a porous polycarbonate membrane that mimics a filtering layer of sinusoidal endothelial cells (see: Dash A, Deering TG, Marukian S, et al. Physiological Hemodynamics and Transport Restore Insulin and Glucagon Responses in a Normal Glucose Milieu in Hepatocytes In Vitro. 73rd Scientific Sessions of the American Diabetes Association, 2013 (Chicago), and U.S. Pat. Nos. 7,811,782 and 9,500,642, and 9,617,521, each of which is incorporated by reference herein in its entirety).

Hepatocytes from propionic academia patients are plated in a collagen gel sandwich on one side of the membrane replicating the polarized orientation found in vivo within the hepatic sinusoids. On the other side, medium is continuously perfused and surface shear rates are applied across a range of physiological values derived from sinusoidal flow rates in vivo while also controlling transport in the system with in- and out-flow tubing to each compartment. Effectively, this creates a flow-based culture system where hepatocytes are shielded from direct effects of flow, as they would be in vivo, but perfusion, nutrient gradients, and interstitial fluid movement are maintained. Under these conditions, human primary hepatocytes in the technology restore in vivo-like morphology, metabolism, transport, and CYP450 activity, and do not de-differentiate.

Hepatocytes are treated with increasing doses of compound 5 (0, 0.1, 0.3, 1, 3, 10, 30, 100 uM) in the HemoShear REVEAL-Tx™ technology from day 5 to day 7. At day 7, islands of cells grown on membrane are cut and placed in 12-well plates and cultured under the same treatment conditions. ¹⁵N—NH₄Cl is added to each well and cells are incubated for 4 hrs. After 4 hrs, cells are washed 2× in saline solution and lysed, scraped and harvested using 80% methanol. ¹⁵N-urea is measured by GCMSMS.

Treatment of primary hepatocytes isolated from livers of propionic acidemia patients with compound 5 resulted in a dose-dependent increase in ¹⁵N-urea. This result indicates that treatment with compound 5 has an effect in improving ureagenesis.

### Example 8

### Reduction of Isovaleryl-CoA in a Primary Hepatocyte Model of Isovaleric Acidemia Following Compound 5 Treatment

Primary hepatocytes are treated with increasing doses of compound 5 (0, 0.1, 0.3, 1, 3, 10, 30, 100 μM) with and without an inhibitor for isovaleryl-CoA dehydrogenase for 30 min. After 30 min the cells are challenged with ¹³C-leucine. At the end of the challenge period, media is removed and the cells are lysed with 70% MeCN and 0.1% TFA containing 100 μM of ethymalonyl-CoA as an internal standard and harvested. Cell lysates are processed for HTMS/MS analysis.

Treatment of primary hepatocytes with compound 5 resulted in a dose-dependent reduction of intracellular isovaleryl-CoA derived from ¹³C-leucine. This indicates that treatment with compound 5 alleviates the primarily metabolic defect (accumulation of isovaleryl-CoA) in a primary hepatocyte model of isovaleric academia.

### Example 9

Reduction of Propionyl-CoA Levels and Accumulation of CoA Ester with Compounds 8 and 9

Primary hepatocytes isolated from livers of propionic acidemia patients are treated with increasing doses of compound 1 (0, 0.1, 0.3, 1, 3, 10, 30, 100 μM) for 30 minutes. After 30 minutes, the cells are challenged with ¹³C-isoleucine (3 mM) for 60 minutes. At the end of the challenge period, media is removed and the cells are lysed with 70% MeCN and 0.1% TFA containing 100 μM of ethymalonyl-CoA as an internal standard and harvested. Cell lysates are processed for HTMS/MS analysis.

Treatment of primary hepatocytes isolated from livers of propionic acidemia patients with compounds 8 or 9 resulted in a dose-dependent reduction of intracellular propionyl-CoA from all sources investigated. This indicates that treatment with compounds 8 or 9 alleviates the primarily metabolic defect (accumulation of propionyl-CoA) in primary hepatocytes of propionic acidemia patients.

### Example 10

Pharmacologic Activity of Compound 5 in Primary Hepatocytes from PA and MMA Patients

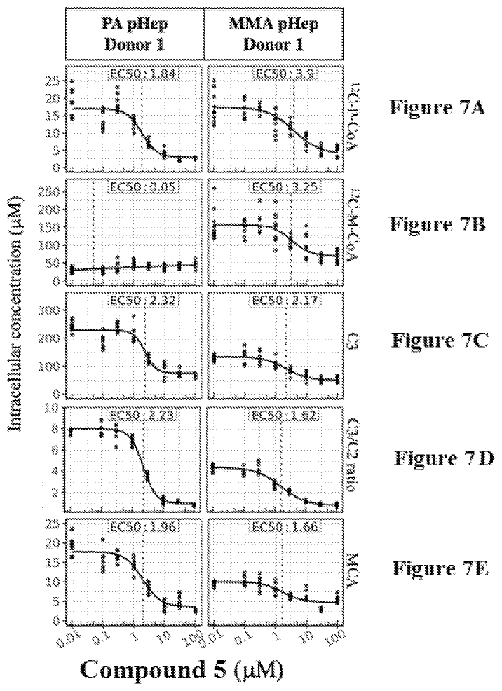

Representative activity data for Compound 5 in primary hepatocytes (pHeps) from PA and MMA donors was demonstrated using the HemoShear REVEAL-Tx™ Technology (FIG. 7). Biomarker levels are normalized to cell counts and cell volume to account for differences in the number of cells plated for each donor. As shown in FIG. 7A, Compound 5 dose-dependently reduced P-CoA in PA and MMA pHeps with EC₅₀ values of 1.84 μM and 3.90 μM, respectively. Compound 5 reduced M-CoA in the MMA pHeps with an EC₅₀ value of 3.25 μM (FIG. 7B). Analysis of PA pHep cell lysates demonstrated an apparent background level of ¹²C-M-CoA of ˜25-50 μM when measured by LC-MS/MS. This is most likely due to the presence of ¹²C-succinyl-CoA in the sample, which has the same mass. In experiments described below, M-CoA is labeled with ¹³C to determine a more accurate percent reduction. Compound 5 reduced the C3 concentration and the C3/C2 ratio with an EC₅₀ similar to that for the reduction in P-CoA (FIGS. 7C, D). MCA is dramatically reduced in both the PA and MMA donors, with an EC₅₀ of 1.96 μM and 1.66 μM, respectively (FIG. 7E).

The summary data for all 3 PA and 3 MMA donors is presented in Table 2. The EC₉₀ values for P-CoA reduction were 18.4±11.3 μM and 36.1±30.1 μM, in PA and MMA pHeps, respectively. Similarly, Compound 5 reduced the concentration of C3 in PA and MMA pHeps with EC₉₀ values of 30.8±26.4 μM and 18.1±16.2 μM, respectively. The EC₉₀ value for reduction in MCA in PA (7.9±3.6 μM) and MMA (7.5±6.4 μM) pHeps was lower than for the other biomarkers. The average EC₉₀ value across all biomarkers was 17.1±13.4 μM, and 30 μM was selected as a fixed concentration to determine the reduction across each biomarker to allow a uniform comparison. The average reduction in P-CoA levels in PA and MMA pHeps at 30 μM was −78.8±10.9% and −74.2±11.6% and for C3 level reductions were −68.9±14.6% and −65.9±10.7%, respectively. The average reduction (expressed as log 2 fold change) in the C3/C2 ratio was −2.1±1.2 in PA pHeps and −2.2±0.2 in MMA pHeps. MCA was reduced by −78.6±12.9% in PA pHeps and −66.7±14.9% in MMA pHeps. Overall, the EC₅₀ values for reduction in biomarker concentrations were consistent across all the biomarkers, suggesting that Compound 5 has a “global” effect on correcting relevant metabolic abnormalities in PA and MMA consistent with the biochemical pathways driving these disease phenotypes and thus supports its therapeutic potential in both disorders (Table 3).

TABLE 3
PA and MMA Biomarker Levels and EC₅₀ and EC₉₀ Values of Compound 5 in the
HemoShear Technology

| | Disease | % change | Curve | | |
|---|---|---|---|---|---|
| Biomarker | pHeps | at 30 μM | count | EC₅₀ μM | EC₉₀ μM |
| ¹²C-Propionyl-CoA | PA | −78.8 ± 10.9 | 6 | 1.9 ± 1 | 18.4 ± 11.3 |
| (P-CoA) | MMA | −74.2 ± 11.6 | 5 | 2.1 ± 1.5 | 36.1 ± 30.1 |
| ¹²C-Methylmalonyl- | PA | NA | NA | NA | NA |
| CoA (M-CoA) | MMA | −55 ± 6.6 | 3 | 2 ± 1.2 | 12.6 ± 12.9 |
| ¹²C-Propionyl- | PA | −69.4 ± 16.2 | 5 | 2.6 ± 1.9 | 32.1 ± 29.3 |
| Carnitine (C3) | MMA | −65.9 ± 10.7 | 4 | 1.4 ± 0.5 | 18.1 ± 16.2 |
| ¹²C-Acetyl- | PA | 54 ± 80.6 | 3 | 4.8 ± 0.2 | 20.7 ± 13.1 |
| Camitine (C2) | MMA | 44.6 ± 26.8 | 3 | 4.3 ± 2.2 | 31.3 ± 26.9 |
| 2-Methylcitric | PA | −78.6 ± 12.9 | 5 | 1.1 ± 0.5 | 7.9 ± 3.6 |
| acid (2MC) | MMA | −66.7 ± 14.9 | 5 | 0.8 ± 0.5 | 7.5 ± 6.4 |

Values are average ± standard deviation
NA—Not applicable

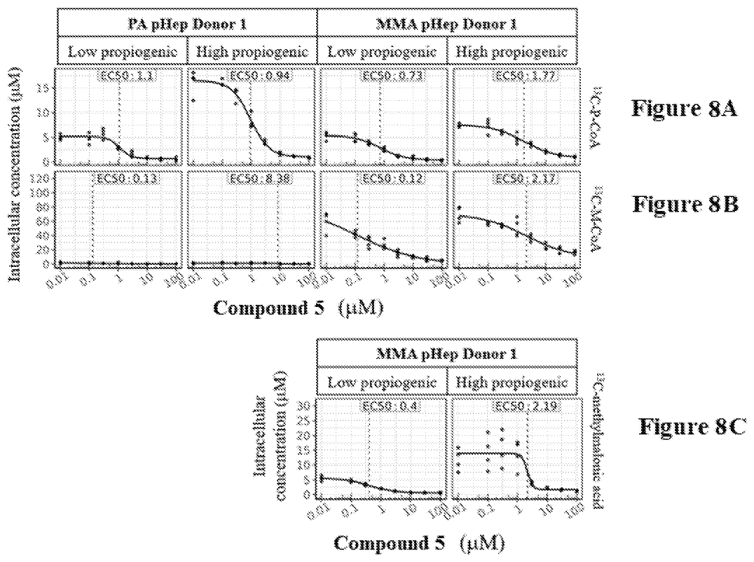

The activity of Compound 5 in primary hepatocytes (pHeps) from PA and MMA donors was also demonstrated using static cell culture experiments. Unlike the HemoShear Technology, this assay is carried out without constant perfusion, utilizing cell culture media that was customized to mimic plasma levels of propiogenic sources (amino acids and ketoacids) in PA and MMA patients during periods of relative metabolic stability (low propiogenic media) and acute metabolic crisis (high propiogenic media). PA and MMA pHeps, in static cell culture, were treated with Compound 5 (concentrations ranging from 0.1 μM to 100 μM) for 30 minutes in low propiogenic media, followed by either a continuation of low propiogenic media or a switch to the high propiogenic media for 1 hour. The media used during the 1-hour incubation contained propiogenic SIL amino acids and ketoacids which are metabolized into labeled P-CoA and M-CoA in the cells. The SIL amino acids and ketoacids were a mix of ¹³C and MeD8 labelling, but their catabolism produced a SIL P-CoA (denoted as ¹³C-P-CoA for simplicity) with the same mass, independent of the type of SIL (also true for ¹³C-M-CoA). Representative data shown in FIG. 8 illustrate that PA pHeps had a more robust increase in ¹³C-P-CoA levels in high propiogenic media, while MMA pHeps had a slight increase in ¹³C-P-CoA, no change in ¹³C-M-CoA, and an increase in methylmalonic acid (FIGS. 8A-8C).

The EC₅₀ values for Compound 5-dependent reduction in ¹³C-P-CoA and ¹³C-M-CoA were similar and independent of low vs high propiogenic media condition (Table 4). The average EC₉₀ value across all biomarkers is 11±9.6 μM. At the dose of 30 μM selected for this calculation (as described above), the percent reduction in ¹³C-P-CoA in PA and MMA pHeps exposed to low propiogenic media was −76.4±12.6% and −77.6±9.8%, respectively. When PA and MMA pHeps were exposed to high propiogenic sources to mimic a metabolic crisis, Compound 5 reduced ¹³C-P-CoA by −85.3±9.1% in PA pHeps and −75.9±7.3% in MMA pHeps. The reduction in ¹³C-M-CoA in MMA pHeps appeared to be somewhat greater under these conditions (low propiogenic: −76.5±13.2%; high propiogenic: −73±5.8%) compared to the ¹²C-M-CoA values measured in the HemoShear Technology (−55±6.6% reduction) (Table 2; Table 3). It is hypothesized that this difference is due to the lower background in ¹³C-M-CoA compared to ¹²C-M-CoA (FIG. 7B vs. FIG. 8B). The EC₉₀ values for reduction of P-CoA and M-CoA are closely matched in the different experimental designs and media formulations. These results indicate that the metabolic pathways do not deteriorate during the very short duration of the static culture experiments.

TABLE 4
PA and MMA Biomarker Levels and EC₅₀ and EC₉₀ Values of
Compound 5 in Low and High Propiogenic Media in Static Cell Culture

| | Disease | | % change at | Curve | | |
|---|---|---|---|---|---|---|
| Biomarker | pHeps | Media | 30 μM | count | EC₅₀ μM | EC₉₀ μM |
| ¹³C-Propionyl-CoA | PA | low | −76.4 ± 12.6 | 5 | 2 ± 1.8 | 6.9 ± 5.4 |
| (¹³C-P-CoA) | MMA | propiogenic | −77.6 ± 9.8 | 6 | 1.3 ± 0.6 | 15.7 ± 18 |
| ¹²C-Methylmalonyl-CoA | PA | | NA | NA | NA | NA |
| (¹³C-M-CoA) | MMA | | −76.5 ± 13.2 | 5 | 2.9 ± 1.6 | 30.9 ± 16.2 |
| ¹³C-Methymalonic acid | PA | | NA | NA | NA | NA |
| | MMA | | −83.2 ± 5 | 3 | 1.2 ± 1.3 | 5.2 ± 3.3 |
| ¹³C-Propionyl-CoA | PA | high | −85.3 ± 9.1 | 6 | 1.7 ± 1 | 11.6 ± 7.4 |
| (¹³C-P-CoA) | MMA | propiogenic | −75.9 ± 7.3 | 6 | 1.6 ± 0.8 | 48.2 ± 47.5 |
| ¹²C-Methylmalonyl-CoA | PA | | NA | NA | NA | NA |
| (¹³C-M-CoA) | MMA | | −73 ± 5.8 | 4 | 2.7 ± 1.6 | 21.2 ± 2 |
| ¹³C-Methymalonic acid | PA | | NA | NA | NA | NA |
| | MMA | | −91 ± 4 | 6 | 1.1 ± 0.9 | 4.6 ± 3 |

Values are average ± standard deviation
NA-Not applicable

### Example 11

### Proposed Mechanism of Action of Compound 5 in Treating MMA and PA

Without being bound by any particular theory, it is believed the mechanism of action for Compound 5 involves the metabolism of Compound 5 in a similar manner to that of small to medium chain fatty acids. For example, Compound 5 can be biotransformed into 2,2-dimethylbutyryl-CoA, also referred to as Compound 5-CoA. This reaction utilizes CoASH. The subsequent metabolism of Compound 5-CoA by β-oxidation would be reduced because Compound 5 does not a have a proton on the alpha carbon, which prevents it from being a substrate for an acyl-CoA dehydrogenase. It is hypothesized that Compound 5 drives a redistribution of the acyl-CoA pools resulting in a reduction of intracellular levels of toxic P-CoA and M-CoA, along with a concomitant lowering of the C3/C2 acyl carnitine ratio and related organic acid metabolites (methylmalonic acid and MCA). This effect on P-CoA levels may be the result of either slowed production or enhanced clearance or a combination of these effects.

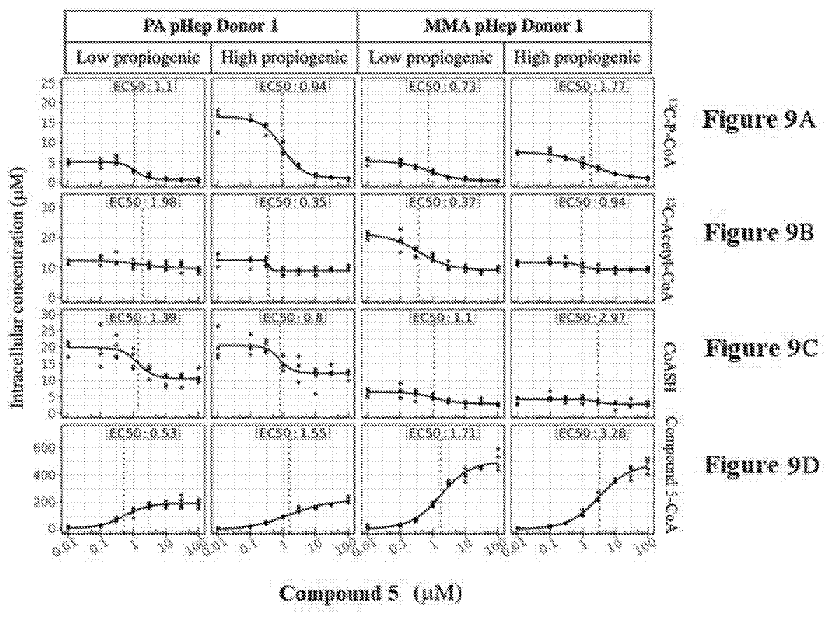

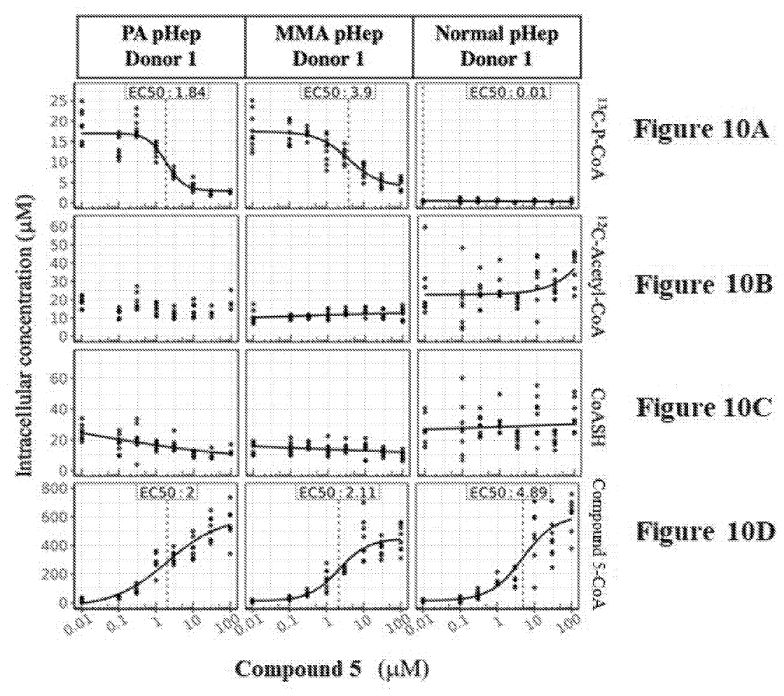

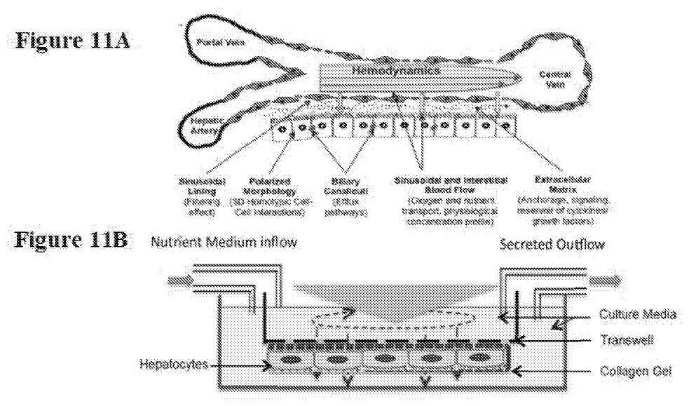

In representative data from PA and MMA pHeps (FIG. 9D; FIG. 10D), the formation of Compound 5-CoA is dose-dependent and similar, whether cells were exposed to Compound 5 over 1.5 hours or 6 days. Importantly, the EC₅₀ value for Compound 5-CoA production correlates with the EC₅₀ value for reduction of P-CoA (FIG. 9A and FIG. 9D; FIG. 10A and FIG. 10D).

In PA and MMA pHeps, where P-CoA and M-CoA levels are very high, there is a dramatic reduction in P-CoA and M-CoA pools with Compound 5 treatment (Table 2 and Table 3). Changes observed with other acyl-CoAs are not as dramatic, suggesting a target specificity that is relevant to the metabolites that accumulate in PA and MMA. The effect of Compound 5 on levels of acetyl-CoA were measured in PA and MMA pHeps in acute static experiments and in PA, MMA and normal pHeps following chronic exposures in the HemoShear Technology (FIG. 9B; FIG. 10B). In static pHeps, there is a partial dose-dependent reduction in acetyl-CoA with acute exposure to Compound 5 (FIG. 9B). Overall, there was significant variability in acetyl-CoA levels following Compound 5 treatment in the HemoShear Technology (FIG. 10B; Table 4). The data illustrate that acetyl-CoA levels were increased in PA, MMA, and normal pHeps, but there was a high SD across the percent change. Although the acetyl-CoA data from the HemoShear Technology were inconclusive, they do not show a decrease in acetyl-CoA as observed in static cell culture experiments (Table 4; Table 5). This may suggest that with Compound 5 treatment over time, acetyl-CoA is not a preferentially targeted acyl-CoA compared P-CoA and M-CoA.

TABLE 5
Compound 5 Pharmacology in the HemoShear Technology

| | Disease | Percent change | Curve | | |
|---|---|---|---|---|---|
| Analyte | pHeps | at 30 μM | count | EC₅₀ μM | EC₉₀ μM |
| 12C-Acetyl- | PA | 54 ± 80.6 | 3 | 2.3 ± 1.9 | 14.4 ± 13.6 |
| CoA | MMA | 13.5 ± 24.9 | 0 | NC | NC |
| | Normal | 20 ± 2.9 | 0 | NC | NC |
| CoASH | PA | −31.3 ± 24.2 | 2 | 11.6 ± 1.2 | 29.8 ± 24.3 |
| | MMA | −22.3 ± 22.3 | 2 | 14.1 ± 8.4 | 23.8 ± 20.2 |
| | Normal | −35.2 ± 3 | 0 | NC | NC |
| Compound 5- | PA | NC | 6 | 3 ± 1.5 | 47.1 ± 39.7 |
| CoA | MMA | NC | 6 | 3.5 ± 1.7 | 52.4 ± 45.6 |
| | Normal | NC | 2 | 8.3 ± 8 | 26.1 ± 3.1 |

Values are average ± standard deviation
NC—Value could not he calculated

To further evaluate the hypothesis that Compound 5 drives a redistribution of the acyl-CoA pools resulting in a reduction in P-CoA and M-CoA, the levels of CoASH were measured in the PA and MMA disease models. In 1.5 hour long static experiments utilizing PA and MMA pHeps, with either low and high propiogenic media, Compound 5 partially reduced CoASH levels with an EC₅₀ value similar to the EC₅₀ values for the reduction in P-CoA and production of Compound 5-CoA (FIG. 9C, Table 6). The effect of Compound 5 on CoASH is less pronounced in PA and MMA pHeps exposed to Compound 5 for 6 days in the HemoShear Technology (FIG. 10C; Table 4). Though in general there is a trend towards a slight decrease in CoASH with increasing dose of Compound 5, the change is statistically significant in less than half of the individual PA and MMA donors. Because many of the curves did not pass quality control, the EC₅₀/EC₉₀ values could be calculated for only 1 PA and 1 MMA donor (Table 4). Of note, the calculated EC₅₀ value for CoASH is 10-fold higher than the EC₅₀ value for the reduction in disease biomarkers (Table 2, Table 4). The Compound 5 mechanism of action is not unique to PA and MMA pHeps. Normal pHeps exposed to Compound 5 in the HemoShear Technology produce Compound 5-CoA and show a reduction in CoASH that is similar to PA and MMA pHeps (FIG. 10, Table 4). The mechanism of action is thought to be consistent regardless of the experimental condition. Results suggest that pHeps chronically exposed to Compound 5 treatment in the HemoShear technology either recover or adapt from the larger changes in CoASH levels observed following acute Compound 5 exposures in static pHeps. Thus, in the more physiologically relevant system, changes in CoASH are slight compared to the robust reductions in P-CoA and M-CoA.

TABLE 6
Compound 5 Pharmacology in Static Culture

| | | | Percent | | | |
|---|---|---|---|---|---|---|
| | Disease | | change | Curve | | |
| Analyte | pHeps | Media | (30 μM) | count | EC₅₀ μM | EC₉₀ μM |
| ¹²C-Acetyl-CoA | PA | low | −36.6 ± 13.5 | 4 | 0.4 ± 0.2 | 1.6 ± 1.3 |
| | MMA | propiogenic | −35.5 ± 18.1 | 6 | 2.5 ± 2.6 | 28.4 ± 52 |
| CoASH | PA | | −55.2 ± 7.1 | 5 | 0.9 ± 0.3 | 9.1 ± 5.8 |
| | MMA | | −47.7 ± 13.4 | 5 | 2.3 ± 1.6 | 14.2 ± 13.5 |
| Compound 5-CoA | PA | | NC | 5 | 1.4 ± 0.6 | 15.3 ± 11.3 |
| | MMA | | NC | 6 | 2 ± 0.7 | 18 ± 15.7 |
| ¹²C-Acetyl-CoA | PA | high | −27 ± 3.4 | 3 | 0.7 ± 0.5 | 1.9 ± 1.3 |
| | MMA | propiogenic | −14.6 ± 12.6 | 7 | 1.4 ± 1.4 | 6.4 ± 7.1 |
| CoASH | PA | | −48 ± 14.3 | 4 | 2.1 ± 1.6 | 11.1 ± 13 |
| | MMA | | −28.7 ± 7.3 | 2 | 13.1 ± 14.8 | 65.6 ± 81.2 |
| Compound 5-CoA | PA | | NC | 6 | 2.6 ± 1.6 | 42 ± 45.8 |
| | MMA | | NC | 6 | 5.4 ± 2.8 | 80.1 ± 57 |

Values are average ± standard deviation
NC-Value could not be calculated

CoA sequestration has been proposed to be associated with toxicity in many disorders of intermediary metabolism, including PA and MMA. It is hypothesized that sequestration of CoASH into accumulating P-CoA and M-CoA leads to a reciprocal decrease in acetyl-CoA and/or CoASH; however, the idea has little to no supporting evidence due to the inability to measure and study tissue acyl-CoA and CoASH levels in humans. While some effect on acetyl-CoA and CoASH was observed, particularly in static culture conditions, these effects were not as pronounced as those observed on other metabolites.

### Conclusions

The studies described herein demonstrated that Compound 5 reduces the toxic metabolites P-CoA, M-CoA, C3, MCA, and methylmalonic acid (MMA only) in pHep-based disease models of PA and MMA. Overall, the EC₉₀ values for reduction of biomarker concentrations were consistent across all the biomarkers, suggesting that Compound 5 has an effect on correcting relevant metabolic abnormalities in PA and MMA that is consistent with the biochemical pathways thought to underlie the disease pathology and thus supports its therapeutic potential in both disorders. Since the compounds of the disclosure are able to form CoA esters, these compounds can also treat diseases characterized by the buildup of toxic levels of the metabolites described herein by redistribution of the acyl-CoA pools.

### INCORPORATION BY REFERENCE

It should also be understood that all patents, publications, journal articles, technical documents, and the like, referred to in this application, are hereby incorporated by reference in their entirety and for all purposes.

The various embodiments described above and throughout the specification can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### Embodiments of the Disclosure

A1. A method of treating an organic acidemia in a subject in need thereof, comprising: administering a compound of Formula (I), or an ester or pharmaceutically acceptable salt thereof, to the subject,

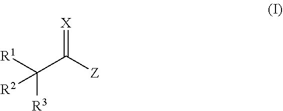

wherein:
X is O, NH, or S;
Z is OR⁴, NR⁴R⁴, SR⁴, halogen, or a leaving group;
each of R¹, R² and R³ are independently H, halogen, alkyl, alkenyl, alkynyl, carbocyclyl, carbocyclylalkyl, heterocyclyl, heterocyclylalkyl, provided that at least one of R₁, R² and R³ is not H;
or any two of R¹, R² and R³ taken together with the carbon atom to which they are attached forms a carbocyclyl or heterocyclyl;
each R⁴ is independently H, alkyl, haloalkyl, alkenyl, alkynyl, carbocyclyl, carbocyclylalkyl, heterocyclyl, heterocyclylalkyl, —C(O)R⁵, —SO₂R⁵, —P(O)OR⁵)₂, or

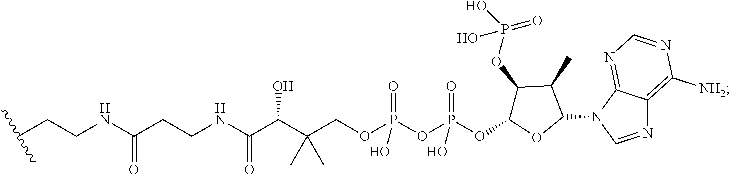

R⁵ is alkyl, haloalkyl, alkenyl, alkynyl, carbocyclyl, carbocyclylalkyl, heterocyclyl, or heterocyclylalkyl;
wherein the administration of the composition reduces at least one metabolite that would otherwise accumulate in an organic acidemia patient, thereby treating the organic acidemia.
   A2. The method of embodiment A1, wherein X is O.
   A3. The method of embodiment A1 or A2, wherein Z is OR⁴.
   A4. The method of any one of embodiments A1-A3, wherein each of R¹, R² and R³ are independently H, halogen, alkyl, alkenyl, alkynyl, carbocyclyl, carbocyclylalkyl, heterocyclyl, or arylalkyl, provided that no more than one of R¹, R² and R³ is H.
   A5. The method of any one of embodiments A1-A4, any two of R¹, R² and R³ taken together with the carbon atom to which they are attached forms a carbocyclyl or heterocyclyl.
   A6. The method of any one of embodiments A1-A5, wherein two of R¹, R² and R³ are alkyl.
   A7. The method of any one of embodiments A1-A6, wherein the alkyl is a C₁₋₆ alkyl, the alkenyl is a C₂₋₆ alkenyl, the alkynyl is a C₂₋₆ alkynyl, the carbocyclyl is a C₃₋₁₂ cycloalkyl or a C₆₋₁₂ aryl, and the heterocyclyl is a C₃₋₁₂ heterocyclyl.
   A8. The method of any one of embodiments A1-A7, wherein R⁴ is independently H, alkyl, alkenyl, alkynyl, carbocyclyl, or carbocyclylalkyl.
   A9. The method of any one of embodiments A1-A7, wherein R⁴ is independently H, alkyl, or, carbocyclyl.
   A10. The method of any one embodiments A1-A9, wherein the compound of Formula (I) is a compound from Table 1A or Table 1B.
   A11. The method of any one of embodiments A1-A10, wherein the compound of Formula (I) is a compound of Formula (IA) having the structure:

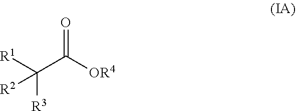

wherein:

each of R¹, R² and R³ is independently H, alkyl, carbocyclyl, or carbocyclylalkyl, provided that at least one of R¹, R² and R³ is not H; and

R⁴ is H or alkyl.

A12. The method of embodiment A11, wherein each of R¹, R² and R³ is independently H, alkyl, or carbocyclyl, provided that at least one of R¹, R² and R³ is not H.
A13. The method of embodiment A11 or A12, wherein at least one of R¹, R² and R³ is alkyl.
A14. The method of embodiment A11 or A12, wherein at least two of R¹, R² and R³ are alkyl.
A15. The method of any one of embodiments A11-A14, wherein the alkyl is a C₁₋₆alkyl.
A16. The method of any one of embodiments A11-A15, wherein the alkyl is selected from the group consisting of methyl, ethyl, n-propyl, n-butyl, and t-butyl.
A17. The method of any one of embodiments A11-A16, wherein one of R¹, R² and R³ is carbocyclyl.
A18. The method of any one of embodiments A11-A17, wherein the carbocyclyl is a cyclopropyl.
A19. The method of any one of embodiments, A11-A13, wherein R¹ is H, R² is H, methyl, ethyl, or n-propyl, and R³ is ethyl, n-propyl, t-butyl, or cyclopropyl.
A20. The method of any one of embodiments A11-A16, wherein R¹ and R² are methyl, and R³ is selected from the group consisting of methyl, ethyl, in-propyl, n-butyl, and cyclopropyl.
A21. The method of any one of embodiments A11-A16, wherein R¹ and R² are methyl, and R³ is ethyl.
A22. The method of any one of embodiments A11-A21, wherein R⁴ is alkyl.
A23. The method of embodiment A22, wherein the alkyl is a C₁₋₄ alkyl.
A24. The method of embodiment A23, wherein the C₁₋₄ alkyl is selected from the group consisting of methyl, ethyl, n-propyl, n-butyl, or t-butyl.
A25. The method of any one of embodiments A11-A21, wherein R⁴ is H.
A26. The method of embodiment A1-A10, wherein the compound of Formula (I) is a compound of Formula (II) having the structure:

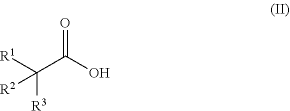

wherein:
each of R¹, R² and R³ is independently H, halogen, alkyl, alkenyl, alkynyl, carbocyclyl, carbocyclylalkyl, heterocyclyl, or heterocyclylalkyl, provided that at least one of R¹, R² and R³ is not H:
or any two of R¹, R² and R³ taken together with the carbon atom to which they are attached forms a carbocyclyl or heterocyclyl.
   A27. The method of embodiment A26, wherein when each of R¹, R² and R³ are independently H, halogen, alkyl, alkenyl, alkynyl, carbocyclyl, carbocyclylalkyl, heterocyclyl, or arylalkyl, and no more than one of R¹, R² and R³ is H.
   A28. The method of embodiment A26 or A27, wherein any two of R¹, R² and R³ taken together with the carbon atom to which they are attached forms a carbocyclyl, or heterocyclyl; and wherein the remaining R¹, R² and R³ is H, halogen, alkyl, alkenyl, alkynyl, carbocyclyl, carbocyclylalkyl, heterocyclyl, heterocyclylalkyl or arylalkyl.
   A29. The method of any one of embodiments A26-A28, wherein the alkyl is a C₁₋₆ alkyl, the alkenyl is a C₂₋₆ alkenyl, the alkynyl is a C₂₋₆ alkynyl, the alkoxy is an O—C₁₋₆ alkyl, the carbocyclyl is a C₃₋₁₂ cycloalkyl or a C₆₋₁₂ aryl, and the heterocyclyl is a C₃₋₁₂ heterocyclyl.
   A30. The method of any one of embodiments A26-A29, wherein each of R¹, R² and R³ is independently H, alkyl, or carbocyclyl, provided that at least one of R¹, R² and R³ is not H.
   A31. The method of any one of embodiments A26-A30, wherein at least one of R¹, R² and R³ is alkyl.
   A32. The method of any one of embodiments A26-A30, wherein at least two of R¹, R² and R³ are alkyl.
   A33. The method of any one of embodiments A26-A32, wherein the alkyl is a C₁₋₆alkyl.
   A34. The method of any one of embodiments A26-A33, wherein the alkyl is selected from the group consisting of methyl, ethyl, n-propyl, n-butyl, and t-butyl.
   A35. The method of any one of embodiments A26-A34, wherein one of R¹, R² and R³ is carbocyclyl.
   A36. The method of any one of embodiments A26-A35, wherein the carbocyclyl is a cyclopropyl.
   A37. The method of any one of embodiments, A26-A30, wherein R¹ is H, R² is H, methyl, ethyl, or n-propyl, and R³ is ethyl, n-propyl, t-butyl, or cyclopropyl.
   A38. The method of any one of embodiments A26-A34, wherein R¹ and R² are methyl, and R³ is selected from the group consisting of methyl, ethyl, n-propyl, n-butyl, and cyclopropyl.
   A39. The method of any one of embodiments A26-A34, wherein R¹ and R² are methyl, and R³ is ethyl.
   A40. The method of any one of embodiments 26-39, wherein the compound of Formula (I) is selected from Table 1A or Table 1B.
   A41. The method of any one of embodiments A1-A30, wherein the compound of Formula (I) is 2,2-dimethylbutyric acid or a pharmaceutically acceptable salt, ester, solvate, or metabolite thereof having the structure:

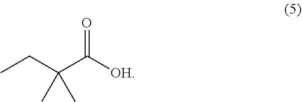

A42. The method of any one of embodiments A1-A41, wherein the organic acidemia is propionic acidemia.
A43. The method of any one of embodiments A1-A41, wherein the organic acidemia is methylmalonic acidemia.
A44. The method of any one of embodiments A1-A41 wherein the organic acidemia is an isovaleric acidemia.
A45. The method of any one of embodiments A1-A44, wherein the compound is present in a pharmaceutical composition that comprises at least one pharmaceutically acceptable excipient.
A46. The method of any one of embodiments A1-A11, wherein when R¹ is H, X is O and Z is OH, each of R² and R³ are not propyl, i.e., the compound is not

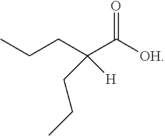

A47. The method of any one of embodiments A1-A11, wherein when X is O and Z is OH, any two of R¹, R² and R³ taken together with the carbon atom to which they are attached are not benzyl substituted at the 3 position with a 1,2,4-oxadiazole.
A48. The method of any one of embodiments A1-A11, wherein when R¹ is H, each of R² and R³ are not propyl.
A49. The method of embodiment A1-A11, wherein any two of R¹, R² and R³ taken together with the carbon atom to which they are attached are not benzyl substituted at the 3 position with a 1,2,4-oxadiazole.
B1. A method of treating an organic acidemia in a subject in need thereof, comprising:

administering 2,2-dimethylbutyric acid, or a pharmaceutically acceptable salt, ester, or metabolite thereof, to the subject,

thereby reducing at least one metabolite that would otherwise accumulate in an organic acidemia patient, thereby treating the organic acidemia.

B2. The method of embodiment B1, wherein the organic acidemia is propionic acidemia.
B3. The method of embodiment B1, wherein the organic acidemia is methylmalonic acidemia.
B4. A method of treating propionic acidemia in a subject in need thereof, comprising:

administering 2,2-dimethylbutyric acid, or a pharmaceutically acceptable salt, ester, or metabolite thereof,

thereby levels of at least one metabolite that would otherwise accumulate in a propionic acidemia patient, thereby treating propionic acidemia in the subject.

B5. A method of treating methylmalonic acidemia in a subject in need thereof, comprising:

administering 2,2-dimethylbutyric acid, or a pharmaceutically acceptable salt, ester, or metabolite thereof,

thereby reducing levels of at least one metabolite that would otherwise accumulate in a methylmalonic acidemia patient, thereby treating methylmalonic acidemia in the subject.

B6. A method of reducing propionyl-CoA or methylmalonyl-CoA production in a subject in need thereof, comprising administering an effective amount 2,2-dimethylbutyric acid, or the pharmaceutically acceptable salt, ester, or metabolite thereof, to the subject.
B7. The method of any of embodiments B4-B6, wherein 2,2-dimethylbutyric acid, or the pharmaceutically acceptable salt, ester, or metabolite thereof, is present in a pharmaceutical composition.
B8. The method of any one of embodiments B1-B3 or B7, wherein the pharmaceutical composition comprises at least one pharmaceutically acceptable excipient and an effective amount of 2,2-dimethylbutyric acid, or an ester, metabolite, or pharmaceutically acceptable salt thereof.
B9. The method of any of embodiments A1-B8, wherein the at least one metabolite comprises propionic acid, 3-hydroxypropionic acid, methylcitrate, glycine, or propionylcarnitine, or combinations thereof.
B10. The method of any of embodiments A1-B9, wherein the at least one metabolite comprises 2-ketoisocaproate, isovaleryl-CoA, 3-methylcrotonyl-CoA, 3-methylglutaconyl-CoA, 3-OH-3-methylglutaryl-CoA, 2-keto-3-methylvalerate, 2-methylbutyryl-CoA, tiglyl-CoA, 2-methyl-3-OH-butyryl-CoA, 2-methyl-acetoacetyl-CoA, 2-ketoisovalerate, isobutyryl-CoA, methylacrylyl-CoA, 3-OH-isobutyryl-CoA, 3-OH-isobutyrate, methylmalonic semialdehyde, propionyl-CoA, or methylmalonyl-CoA, or combinations thereof.
B11. The method of any of embodiments A1-B10, wherein the at least one metabolite is reduced by an amount ranging from at least about 1% to about 100%.
B12. A method of treating a metabolic disorder, comprising administering 2,2-dimethylbutyric acid, or an ester, metabolite, or pharmaceutically acceptable salt thereof.
B13. The method of embodiment B12, wherein the metabolic disorder is selected form the group consisting of propionic acidemia, methylmalonic acidemia, mitochondrial short-chain enoyl-CoA hydratase 1 deficiency (OMIM 616277), 3-hydroxyisobutyryl-CoA hydrolase deficiency (OMIM 250620), 3-hydroxyisobutyrate dehydrogenase deficiency, methylmalonate-semialdehyde dehydrogenase deficiency (OMIM 614105), 2-methyl-3-hydroxybutyryl-CoA dehydrogenase deficiency (OMIM 300438), or 3-methylacetoacetyl-CoA thiolase deficiency (OMIM 203750), and combinations thereof.
B14. The method of embodiment B9, wherein the at least one metabolite is reduced by an amount in the range of from about 1% to about 100%.
B15. The method of embodiment B1, wherein the organic acidemia is isovaleric acidemia.

We claim:

1. A method of treating an organic acidemia in a subject in need thereof, comprising:
administering 2,2-dimethylbutyric acid, or a pharmaceutically acceptable salt or ester thereof, to the subject,
thereby reducing at least one metabolite that would otherwise accumulate in an organic acidemia patient, thereby treating the organic acidemia.

2. The method of claim 1, wherein the organic acidemia is propionic acidemia.

3. The method of claim 1, wherein the organic acidemia is methylmalonic acidemia.

4. A method of treating propionic acidemia in a subject in need thereof, comprising:
administering 2,2-dimethylbutyric acid, or a pharmaceutically acceptable salt or ester thereof,
thereby reducing levels of at least one metabolite that would otherwise accumulate in a propionic acidemia patient, thereby treating propionic acidemia in the subject.

5. A method of treating methylmalonic acidemia in a subject in need thereof, comprising:
administering 2,2-dimethylbutyric acid, or a pharmaceutically acceptable salt or ester thereof,
thereby reducing levels of at least one metabolite that would otherwise accumulate in a methylmalonic acidemia patient, thereby treating methylmalonic acidemia in the subject.

6. A method of reducing propionyl-CoA or methylmalonyl-CoA production in a subject in need thereof, comprising administering an effective amount 2,2-dimethylbutyric acid, or the pharmaceutically acceptable salt or ester thereof, to the subject.

7. The method of any one of claims 1-6, wherein 2,2-dimethylbutyric acid, or the pharmaceutically acceptable salt or ester thereof, is present in a pharmaceutical composition.

8. The method of claim 7, wherein the pharmaceutical composition comprises at least one pharmaceutically acceptable excipient and an effective amount of 2,2-dimethylbutyric acid, or an ester or pharmaceutically acceptable salt thereof.

9. The method of any of claims 1-8, wherein the at least one metabolite comprises propionic acid, 3-hydroxypropionic acid, methylcitrate, glycine, or propionylcarnitine, or combinations thereof.

10. The method of any of claims 1-9, wherein at least one metabolite comprises 2-ketoisocaproate, isovaleryl-CoA, 3-methylcrotonyl-CoA, 3-methylglutaconyl-CoA, 3-OH-3-methylglutaryl-CoA, 2-keto-3-methylvalerate, 2-methylbutyryl-CoA, tiglyl-CoA, 2-methyl-3-OH-butyryl-CoA, 2-methyl-acetoacetyl-CoA, 2-ketoisovalerate, isobutyryl-CoA, methylacrylyl-CoA, 3-OH-isobutyryl-CoA, 3-OH-isobutyrate, methylmalonic semialdehyde, propionyl-CoA, or methylmalonyl-CoA, or combinations thereof.

11. The method of any of claims 1-10, wherein the at least one metabolite is reduced by an amount ranging from at least about 1% to about 100%.

12. A method of treating a metabolic disorder, comprising administering 2,2-dimethylbutyric acid, or an ester or pharmaceutically acceptable salt thereof.

13. The method of claim 12, wherein the metabolic disorder is selected form the group consisting of propionic acidemia, methylmalonic acidemia, mitochondrial short-chain enoyl-CoA hydratase 1 deficiency (OMIM 616277), 3-hydroxyisobutyryl-CoA hydrolase deficiency (OMIM 250620), 3-hydroxyisobutyrate dehydrogenase deficiency, methylmalonate-semialdehyde dehydrogenase deficiency (OMIM 614105), 2-methyl-3-hydroxybutyryl-CoA dehydrogenase deficiency (OMIM 300438), or 3-methylacetoacetyl-CoA thiolase deficiency (OMIM 203750), 3-hydroxy-3-methylglutaric aciduria and combinations thereof.

14. The method of claim 9, wherein the at least one metabolite is reduced by an amount in the range of from about 1% to about 100%.

15. The method of claim 1, wherein the organic acidemia is isovaleric acidemia.

16. The method of any one of claims 1-15, wherein the pharmaceutically acceptable salt of 2,2-dimethylbutyric acid is a sodium salt.

17. A method of treating an organic acidemia in a subject in need thereof, comprising: administering a compound of Formula (IA), or an ester or pharmaceutically acceptable salt thereof, to the subject, 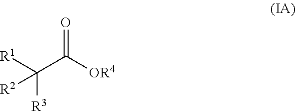
wherein:
each of R¹, R² and R³ is independently H, alkyl, carbocyclyl, or carbocyclylalkyl, provided that at least one of R¹, R² and R³ is not H; and
R⁴ is H alkyl, or carnitine.

18. The method of claim 17, wherein each of R¹, R² and R³ is independently H, alkyl, or carbocyclyl, provided that at least one of R¹, R² and R³ is not H.

19. The method of claim 17 or 18, wherein at least one of R¹, R² and R³ is alkyl.

20. The method of claim 17 or 18, wherein at least two of R¹, R² and R³ are alkyl.

21. The method of any one of claims 17-20, wherein the alkyl is a C₁₋₆ alkyl.

22. The method of any one of claims 17-21, wherein the alkyl is selected from the group consisting of methyl, ethyl, in-propyl, n-butyl, and t-butyl.

23. The method of any one of claims 17-22, wherein one of R¹, R² and R³ is carbocyclyl.

24. The method of any one of claims 17-23, wherein the carbocyclyl is a cyclopropyl.

25. The method of any one of claims, 17-22, wherein R¹ is H, R² is H, methyl, ethyl, or n-propyl, and R³ is ethyl, n-propyl, t-butyl, or cyclopropyl.

26. The method of any one of claims 17-22, wherein R¹ and R² are methyl, and R³ is selected from the group consisting of methyl, ethyl, n-propyl, n-butyl, and cyclopropyl.

27. The method of any one of claims 17-22, wherein R¹ and R² are methyl, and R³ is ethyl.

28. The method of any one of claims 17-27, wherein R⁴ is alkyl.

29. The method of claim 28, wherein the alkyl is a C₁₋₄ alkyl.

30. The method of claim 29, wherein the C₁₋₄ alkyl is selected from the group consisting of methyl, ethyl, n-propyl, n-butyl, or t-butyl.

31. The method of any one of claims 17-27, wherein R⁴ is H.

32. A method of treating a patient with elevated propionyl-CoA or methylmalonyl-CoA with a combination of 2,2-dimethylbutyric acid or pharmaceutically acceptable salt thereof in combination with carnitine

33. A method of treating a patient that has had a liver, kidney or liver and kidney transplant with 2,2-dimethylbutryic acid or pharmaceutically acceptable salt thereof.

34. A method of treating a patient with 2,2-dimethylbutryic acid or pharmaceutically acceptable salt thereof before, after or in combination with mRNA-3927, mRNA-3704 or LB001.

35. A method of treating a patient with 2,2-dimethylbutryic acid or pharmaceutically acceptable salt thereof before, after or during AAV delivered gene therapy that is designed to replace PCC or MUT.

36. A method of treating a patient with 2,2-dimethylbutryic acid or pharmaceutically acceptable salt thereof before, after or during a gene therapy treatment that is designed to replace PCC or MUT.
